SEQUENCE LISTING (19 sequences)

SEQ ID NO: 1 - DNA, 3063 bp, Limulus polyphemus
atggtgcttgcctcctttctcgtgtccggtcttgtgctcggcctcctcgcccaaaagatgcgacccgtgcagtccagaggagtggacctgggcctgtgcgacgatacccggttcgagtgcaaatgcggcgacccgggttacgtgttcaatgtgcctgcgaagcagtgtacctacttttaccgctggcgcccttactgcaagccatgcgacaagcttgaggctaaagatgtgtgccccaagtacaagaggtgccaagaatgccgcgccggcctggattcatgcgtgagctgccccccaaacaaatacggcacttggtgctccggggagtgccagtgcaagaacggcggaatctgcgaccagaggaccggggcctgcacttgccgggaccgctacgagggggtgcattgcgagatcctgcagggttgcccgctgctgcagtccgacccccaagtgcaggaggtcaagaacccccccaacgacccgcagaccatcgactactcctgttcacctgggttcaagctgaagggcgtggcacggattacgtgcctccccaacggacagtggtcatccttcccccctaagtgcattcgggagtgcagcatggtgtcgagcttggaacacggaaaggtcaactccccgagcgccgacctgattgagggggcgacccttcggttctcgtgcgactccccctattacctgatcggtcaggaaaccctgacgtgccagggcaacggccaatggtcggggcagatcccgcagtgtcagaagctcgtgttttgtcccgatttggacccagtgtcccatgccgaacaccaggtcaagatcggactggaacagaaatacggacagttcccccaaggcactgaagtcacttacacttgcaccggaaactacttcctgatgggcctggacaccctcaagtgcaatcccgacggatcctggtcgggcactcagccgtcctgcgtgaaagtggcagacagagaagtgaactgtgatagcaaagctgtggacttcctggacgacgtgggcgaaccggtccgcatccactgcccggccgggtgcagcctgaccgcgggtactgtctggggtaccgccatctatcatgaactttcgtccgtctgccgggccgccattcacgccggaaaggtcccgaatagcggcggtgcagtgcacgtcgtgaacaacggaccctactcggatttcctcgcctcggacttgaatggcattaagtccgacgagctgaagtccctggcccagtccttccggttcgactacgtgtcatccagcacggccggacggaagtcgggctgccctgatggctggttcgaaatcgaagagaattgtgtctacgtgacctcaaagcagagagcctgggaacgggctcagggagtctgcactaacatggctgcccggttggccgtgctcgacaaggatgtgatcccgtcctcgctcactgaaaccctgcggggaaagggactggctaccacttggatcggactgcacaggctcgatgcggacaaccacttcatctgggaactgatggaccgctcctccgtggccctgaacgacagcctgaccttctgggcccctggagagccaggaaacgaaaccaactgcgtgtacctggacatccaggaccaactgcagcctgtctggaaaaccaagtcgtgctttcaaccctcctctttcgtgtgcatgatggacctgagcgataagaacaaggccaagtgcaaggatcccggcccgctcgagaacggtcacgcgaagctgcacgggcagtccatcgacggcttctatgccggttcctccgtgcgctactcgtgtgaagtgttgcactacctgtccggtaccgaaaccgtgtcctgtacctccaacgggacttggtcggccccgaagccacgctgtattaaggtcatcacctgtcagactccgcctgtcccgtcctacgggtccgtggatatcaagcccccgagccggactaactcgattagccgcgtgggctcaccctttctgcggctcccaaggttgccgcttccgctggcccgggcggccggcccgcctccgaagcctagatccgcgcctccgtccaccgtggatttgtccagcaaagtcaagctccctgagggacattacagagtgggatcccaggccatctacacttgcgaatcacgctattacgagctgctcggatcgcaaggcagacgctgtgacagcaacggaaagtggtcgggcagaccggccagctgtattcccgtgtgcgggcggagcgactcgcctcgctcccccttcatcgtgaacggaaactcaaccgaaatcggccagtggccgtggcaggccggaatctctcggtggctggctgatcataacatgtggttcctgcaatgcgggggcgccctgctgaacgagaagtggataatcacagccgctcactgcgtgacatactcggcaaccgccgaaatcatcgatccctcccaattcaagttctacctgggaaagtactaccgggatgactctaaggacgacgattatgtgcaagtccgggaagccattgagatccacgtgaaccctaactacgatccgggaaatctgaacttcgacattgcgctgatccagctgaaaacctccgtggcgctgaccactcgcgtccagccgatctgcctccccactgacctgaccacacgggagaacctgaaggaaggggccctggcagtcgtgaccggatggggactcaacgagaacaacacctactccgagatgatccagcaggccgtgctccctgtggtggcggccagcacctgtgaacagggctaccaggactccggcctcccactgactgtgactgagaacatgttctgtgccgggtacaagcaggggcgctacgacgcgtgttccggcgacagcggcgggccactggtgttcgccgacgactcgaggaccgaccggcgctgggtgctcgagggtattgtgtcctggggatcccccaacggatgcgggaagtccaaccaatacggcggattcaccaaggtcaacgtgttcttgtcctggattcgccagttcatctga

SEQ ID NO: 2 - protein, 995 aa, Limulus polyphemus
RGVDLGLCDDTRFECKCGDPGYVFNVPAKQCTYFYRWRPYCKPCDKLEAKDVCPKYKRCQECRAGLDSCVSCPPNKYGTWCSGECQCKNGGICDQRTGACTCRDRYEGVHCEILQGCPLLQSDPQVQEVKNPPNDPQTIDYSCSPGFKLKGVARITCLPNGQWSSFPPKCIRECSMVSSLEHGKVNSPSADLIEGATLRFSCDSPYYLIGQETLTCQGNGQWSGQIPQCQKLVFCPDLDPVSHAEHQVKIGLEQKYGQFPQGTEVTYTCTGNYFLMGLDTLKCNPDGSWSGTQPSCVKVADREVNCDSKAVDFLDDVGEPVRIHCPAGCSLTAGTVWGTAIYHELSSVCRAAIHAGKVPNSGGAVHVVNNGPYSDFLASDLNGIKSDELKSLAQSFRFDYVSSSTAGRKSGCPDGWFEIEENCVYVTSKQRAWERAQGVCTNMAARLAVLDKDVIPSSLTETLRGKGLATTWIGLHRLDADNHFIWELMDRSSVALNDSLTFWAPGEPGNETNCVYLDIQDQLQPVWKTKSCFQPSSFVCMMDLSDKNKAKCKDPGPLENGHAKLHGQSIDGFYAGSSVRYSCEVLHYLSGTETVSCTSNGTWSAPKPRCIKVITCQTPPVPSYGSVDIKPPSRTNSISRVGSPFLRLPRLPLPLARAAGPPPKPRSAPPSTVDLSSKVKLPEGHYRVGSQAIYTCESRYYELLGSQGRRCDSNGKWSGRPASCIPVCGRSDSPRSPFIVNGNSTEIGQWPWQAGISRWLADHNMWFLQCGGALLNEKWIITAAHCVTYSATAEIIDPSQFKFYLGKYYRDDSKDDDYVQVREAIEIHVNPNYDPGNLNFDIALIQLKTSVALTTRVQPICLPTDLTTRENLKEGALAVVTGWGLNENNTYSEMIQQAVLPVVAASTCEQGYQDSGLPLTVTENMFCAGYKQGRYDACSGDSGGPLVFADDSRTDRRWVLEGIVSWGSPNGCGKSNQYGGFTKVNVFLSWIRQFI

SEQ ID NO: 3 - protein, 1020 aa, Limulus polyphemus
MVLASFLVSGLVLGLLAQKMRPVQSRGVDLGLCDDTRFECKCGDPGYVFNVPAKQCTYFYRWRPYCKPCDKLEAKDVCPKYKRCQECRAGLDSCVSCPPNKYGTWCSGECQCKNGGICDQRTGACTCRDRYEGVHCEILQGCPLLQSDPQVQEVKNPPNDPQTIDYSCSPGFKLKGVARITCLPNGQWSSFPPKCIRECSMVSSLEHGKVNSPSADLIEGATLRFSCDSPYYLIGQETLTCQGNGQWSGQIPQCQKLVFCPDLDPVSHAEHQVKIGLEQKYGQFPQGTEVTYTCTGNYFLMGLDTLKCNPDGSWSGTQPSCVKVADREVNCDSKAVDFLDDVGEPVRIHCPAGCSLTAGTVWGTAIYHELSSVCRAAIHAGKVPNSGGAVHVVNNGPYSDFLASDLNGIKSDELKSLAQSFRFDYVSSSTAGRKSGCPDGWFEIEENCVYVTSKQRAWERAQGVCTNMAARLAVLDKDVIPSSLTETLRGKGLATTWIGLHRLDADNHFIWELMDRSSVALNDSLTFWAPGEPGNETNCVYLDIQDQLQPVWKTKSCFQPSSFVCMMDLSDKNKAKCKDPGPLENGHAKLHGQSIDGFYAGSSVRYSCEVLHYLSGTETVSCTSNGTWSAPKPRCIKVITCQTPPVPSYGSVDIKPPSRTNSISRVGSPFLRLPRLPLPLARAAGPPPKPRSAPPSTVDLSSKVKLPEGHYRVGSQAIYTCESRYYELLGSQGRRCDSNGKWSGRPASCIPVCGRSDSPRSPFIVNGNSTEIGQWPWQAGISRWLADHNMWFLQCGGALLNEKWIITAAHCVTYSATAEIIDPSQFKFYLGKYYRDDSKDDDYVQVREAIEIHVNPNYDPGNLNFDIALIQLKTSVALTTRVQPICLPTDLTTRENLKEGALAVVTGWGLNENNTYSEMIQQAVLPVVAASTCEQGYQDSGLPLTVTENMFCAGYKQGRYDACSGDSGGPLVFADDSRTDRRWVLEGIVSWGSPNGCGKSNQYGGFTKVNVFLSWIRQFI

SEQ ID NO: 4 - DNA, 1203 bp, Limulus polyphemus
atggcctggatctgtgtcattacccttttcgctctcgcctcgtctactctgtccaacaaagtgtcgagggtcggaatcatcttccccaagactcagaacgacaacaagcagtgcacggctaagggtggactcaagggcagctgcaaatccctcactgactgccccgccgtgctggcgacccttaaggacagcttccctgtcgtgtgtagctggaatggccggttccaaccgattgtgtgctgtccggacgccgccgccccttccgtgacaactaccgtgaccactatcgtgccgaccaaggaaactaagatccctagactgcatatcccgggctgcgggaaacggaaagtcaacgtggacatcaccaccatcggacggtccggtagcccgatcctgccgcccattagcacgtcacaggacctgaaggggggccgcggaatcattgccggcggagtggaagcgaagattggggcgtggccctggatggccgcagtgttcgtgaagaactttggcattggaagattccactgcgcgggatccatcatttcgtccaagtacattctgtccgccgcccacgccttcctgatcggcggccgcaagctgacccccacccgcttggccgtgcgcgtgggcggacactacgtcaagatgggccaagagtaccacgtggaagatgtcatcatccaccctgactacgtggaacgggagaactacaacgacatcgctatcatcgtgctgaaggaggagctgaacttcaccgacctcgtgcgcccaatctgcctgccggatccagaggccgtgaccgattcactcaagggtcggagagtgaccgtcgccggttggggggacctcgacttcgcgggcccccggtcccaagtgctgcgggaggtgtccataccagtggtgcctattggcgattgcaacaaggcatatcagaagctcaacaccttggctctgaagaacggaattaccaagaagttcatctgcgccgggctggaagaagggggaaaggacgcgtgccagggcgattcgggtggacccctgatgctggtcaacaactcatcgtggatcgtgaccggagtggtgtccttcggacataagtgcgccgaagaagggtttccgggagtctacaccagggtggtgtcctacctggagtggatcgcaaaggtcaccaattccttggatcagaccgtcactaactaa

SEQ ID NO: 5 - protein, 375 aa, Limulus polyphemus
IIFPKTQNDNKQCTAKGGLKGSCKSLTDCPAVLATLKDSFPVVCSWNGRFQPIVCCPDAAAPSVTTTVTTIVPTKETKIPRLHIPGCGKRKVNVDITTIGRSGSPILPPISTSQDLKGGRGIIAGGVEAKIGAWPWMAAVFVKNFGIGRFHCAGSIISSKYILSAAHAFLIGGRKLTPTRLAVRVGGHYVKMGQEYHVEDVIIHPDYVERENYNDIAIIVLKEELNFTDLVRPICLPDPEAVTDSLKGRRVTVAGWGDLDFAGPRSQVLREVSIPVVPIGDCNKAYQKLNTLALKNGITKKFICAGLEEGGKDACQGDSGGPLMLVNNSSWIVTGVVSFGHKCAEEGFPGVYTRVVSYLEWIAKVTNSLDQTVTN

SEQ ID NO: 6 - protein, 400 aa, Limulus polyphemus
MAWICVITLFALASSTLSNKVSRVGIIFPKTQNDNKQCTAKGGLKGSCKSLTDCPAVLATLKDSFPVVCSWNGRFQPIVCCPDAAAPSVTTTVTTIVPTKETKIPRLHIPGCGKRKVNVDITTIGRSGSPILPPISTSQDLKGGRGIIAGGVEAKIGAWPWMAAVFVKNFGIGRFHCAGSIISSKYILSAAHAFLIGGRKLTPTRLAVRVGGHYVKMGQEYHVEDVIIHPDYVERENYNDIAIIVLKEELNFTDLVRPICLPDPEAVTDSLKGRRVTVAGWGDLDFAGPRSQVLREVSIPVVPIGDCNKAYQKLNTLALKNGITKKFICAGLEEGGKDACQGDSGGPLMLVNNSSWIVTGVVSFGHKCAEEGFPGVYTRVVSYLEWIAKVTNSLDQTVTN

SEQ ID NO: 7 - DNA, 1149 bp, Limulus polyphemus
atgggaatcttgccctcgcccggaatgcctgccctgcttagcctcgtgtcactcctgtccgtcctgctcatgggctgcgtggcctcctcactgggacgccagcggagacagttcgtgttcccggatgacgaagagtcctgtagcaaccggtttaccaacgacgggatctgcaaggacgtgctgaactgccgcgatctgctgcagaagaatgactacaacctcttgaaggaatccatctgcggcttcgaagggatcacccctaaagtctgctgtccaaagcaatccattgtgaaccccatcaccgaagccccgcctaagaccaccactactgagaggcctccgatccggatcccctccaacctcccgaagcagtgcggaaatcggaacatcactaccactaggatcatcggcggacaggaagccactccgggagcctggccgtggatggcagccgtgtacattaagcagggggggattcggtccgtgcagtgcggtggcgcccttgtgaccaacagacacgtgatcaccgcctcacactgcgtcgtgaattccctgggtaccgatgtgatgagggctgatgtgttctccgtgcggctgggagaacataacctgtacagcactaacgactcgtcagacccaatcgacttcgccgtgacgagcgtgaagcaccatgaaaacttcgtgctggccacctacctcaacgacatcgcgatcctgaagctgaacgacactgtcaccttcacacacaagattaagccgatttgcctcccttacgagtccctgcgctatgaggacctcgctatgcgcaaccccttcgtggccggatggggcactaccgcgttcaacggaccgagcagcgctgtcctgagagaagtgcaactcccaatctggggtcacgagccgtgccgccaagcgtacgagaaagacctgaacatcaccaacgtgtatatgtgcgcgggctacgccgacggtggaaaggacgcatgccagggcgattctggcggccccatgatgctgcctgacaagtccgggaacttctacctggtcggaatcgtgtcgttcggaaagaaatgcgccctgcccggctttcccggagtgtacaccaaggtcaccgaatttttggattggattgccgtgaacatggtctaa

SEQ ID NO: 8 - protein, 354 aa, Limulus polyphemus
SSLGRQRRQFVFPDDEESCSNRFTNDGICKDVLNCRDLLQKNDYNLLKESICGFEGITPKVCCPKQSIVNPITEAPPKTTTTERPPIRIPSNLPKQCGNRNITTTRIIGGQEATPGAWPWMAAVYIKQGGIRSVQCGGALVTNRHVITASHCVVNSLGTDVMRADVFSVRLGEHNLYSTNDSSDPIDFAVTSVKHHENFVLATYLNDIAILKLNDTVTFTHKIKPICLPYESLRYEDLAMRNPFVAGWGTTAFNGPSSAVLREVQLPIWGHEPCRQAYEKDLNITNVYMCAGYADGGKDACQGDSGGPMMLPDKSGNFYLVGIVSFGKKCALPGFPGVYTKVTEFLDWIAVNMV

SEQ ID NO: 9 - protein, 382 aa, Limulus polyphemus
MGILPSPGMPALLSLVSLLSVLLMGCVASSLGRQRRQFVFPDDEESCSNRFTNDGICKDVLNCRDLLQKNDYNLLKESICGFEGITPKVCCPKQSIVNPITEAPPKTTTTERPPIRIPSNLPKQCGNRNITTTRIIGGQEATPGAWPWMAAVYIKQGGIRSVQCGGALVTNRHVITASHCVVNSLGTDVMRADVFSVRLGEHNLYSTNDSSDPIDFAVTSVKHHENFVLATYLNDIAILKLNDTVTFTHKIKPICLPYESLRYEDLAMRNPFVAGWGTTAFNGPSSAVLREVQLPIWGHEPCRQAYEKDLNITNVYMCAGYADGGKDACQGDSGGPMMLPDKSGNFYLVGIVSFGKKCALPGFPGVYTKVTEFLDWIAVNMV

SEQ ID NO: 10 - DNA, 3051 bp, Tachypleus tridentatus
atgaagttcctcgtcaacgtcgccctcgtgtttatggtggtgtatattagctacatctatgctcgtggcgtcgatctgggcctgtgcgatgagacccgcttcgaatgtaagtgcggcgaccccggctatgtgttcaacgtccccatgaagcagtgcacctacttctaccgctggcgcccctactgtaagccttgcgatgacctggaagctaaagatatttgccctaagtataagcgctgccaagaatgtaaggctggtctggattcctgcgtcacctgtccccctaacaagtatggcacctggtgttccggtgaatgccagtgcaaaaacggcggcatctgcgatcagcgtactggcgcttgcacctgccgtgatcgctacgagggcgcccactgtgaaattctgaaaggctgtcctctgctgccttccgatagccaagtgcaggaggtgcgtaacccccctgacaaccctcagaccatcgattatagctgctcccccggcttcaagctgaagggcgtggcccgtattagctgtctgcccaacggtcagtggagctcctttccccccaagtgcatccgtgaatgtgccaaggtctcctcccccgaacacggcaaggtcaacgctccctccggcaacatgatcgaaggcgccaccctccgtttttcctgcgactccccttactacctcatcggccaggagaccctgacttgtcagggcaatggccagtggagcggtcaaatccctcagtgcaagaagctggtgttctgtcctgacctcgatcccgtgaaccacgctgagcaccaagtgaagatcggcgtggaacaaaagtacggtcagttccctcagggcactgaggtcacttacacttgctccggcaactacttcctgatgggcttcaacaccctgaaatgtaaccccgacggttcctggtccggttcccagccctcctgcgtgaaagtcgctgaccgtgaagtcgactgtgacagcaaagccgtggacttcctggacgacgtgggcgagcctgtgcgtattcactgtcccgctggctgtagcctgactgccggtaccgtgtggggtactgctatctaccacgagctctccagcgtgtgccgcgctgctatccacgctggcaagctgcctaacagcggcggtgctgtccacgtggtgaacaacggtccctactccgacttcctcggtagcgacctgaacggtatcaaaagcgaggaactgaagtccctggcccgtagctttcgcttcgactacgtgtcctcctccaccgctggccgtagcggctgtcctgatggctggtttgaggtggaagagaattgtgtgtacgtgacctccaagcagcgtgcttgggagcgtgctcagggtgtctgtaccaatatggccgcccgtctggctgtcctcgataaggatctcatcccttcctccctcaccgagactctgcgcggtaagggcctgaccactacctggatcggtctccaccgcctggacgctgagaagcctttcgtctgggagctcatggaccgcagcaacgtggtgctgaacgacaacctgaccttctgggccagcggcgagcctggcaatgaaaccaactgcgtgtacctggacatccgtgatcagctccagcccgtgtggaagaccaagagctgcttccagccctccagctttgcttgcatgatggatctctccgaccgcaacaaggctaaatgtgatgatcccggccccctggagaacggccatgccactctgcatggccagagcatcgacggtttctacgctggttcctccatccgctactcctgcgaagtgctccactacctgtccggcaccgagaccgtgacctgtactaccaacggtacttggagcgctcctaaaccccgctgtatcaaggtgattacctgtcagaaccctcctgtccccagctacggctccgtggaaatcaagccccccagccgtactaatagcatcagccgtgtcggttcccctttcctgcgtctgcctcgtctgcccctgcctctcgctcgcgctgctaagcctccccctaagccccgttccagccagcctagcactgtggatctcgctagcaaagtgaaactccccgagggtcactatcgtgtgggctcccgcgccatttacacctgcgagagccgctactacgagctcctgggcagccagggtcgtcgttgcgacagcaacggtaactggagcggtcgccctgccagctgcattcccgtgtgcggtcgcagcgactcccctcgcagccctttcatctggaacggcaacagcaccgagatcggtcagtggccctggcaagctggcattagccgctggctcgctgatcacaacatgtggttcctccaatgtggcggcagcctgctgaacgagaagtggatcgtgactgccgctcactgcgtgacctacagcgccaccgctgagatcatcgacccctcccaatttaagatctacctcggcaagtactaccgcgacgatagccgcgacgacgattatgtgcaagtccgcgaagctctcgaaattcacgtgaaccctaactacgatcccggtaacctgaacttcgacattgccctgatccagctgaagacccctgtgaccctcaccactcgcgtccagcctatctgcctccccaccgacattaccactcgcgaacatctgaaagaaggtaccctggccgtcgtgaccggctggggcctcaacgaaaacaacacttacagcgagatgatccagcaggctgtgctgcctgtcgtggctgctagcacctgtgaggaaggctacaaagaggccgatctccccctgaccgtcaccgagaacatgttttgcgctggttacaaaaagggccgctacgacgcctgcagcggtgatagcggtggccctctcgtgttcgccgatgattcccgtaccgaacgtcgctgggtgctcgagggcattgtgtcctggggtagcccttccggctgtggcaaggctaaccaatacggcggcttcaccaaggtgaacgtgttcctgtcctggattcgtcagttcatctaatga

SEQ ID NO: 11 - protein, 994 aa, Tachypleus tridentatus
RGVDLGLCDETRFECKCGDPGYVFNVPMKQCTYFYRWRPYCKPCDDLEAKDICPKYKRCQECKAGLDSCVTCPPNKYGTWCSGECQCKNGGICDQRTGACTCRDRYEGAHCEILKGCPLLPSDSQVQEVRNPPDNPQTIDYSCSPGFKLKGVARISCLPNGQWSSFPPKCIRECAKVSSPEHGKVNAPSGNMIEGATLRFSCDSPYYLIGQETLTCQGNGQWSGQIPQCKKLVFCPDLDPVNHAEHQVKIGVEQKYGQFPQGTEVTYTCSGNYFLMGFNTLKCNPDGSWSGSQPSCVKVADREVDCDSKAVDFLDDVGEPVRIHCPAGCSLTAGTVWGTAIYHELSSVCRAAIHAGKLPNSGGAVHVVNNGPYSDFLGSDLNGIKSEELKSLARSFRFDYVSSSTAGRSGCPDGWFEVEENCVYVTSKQRAWERAQGVCTNMAARLAVLDKDLIPSSLTETLRGKGLTTTWIGLHRLDAEKPFVWELMDRSNVVLNDNLTFWASGEPGNETNCVYLDIRDQLQPVWKTKSCFQPSSFACMMDLSDRNKAKCDDPGPLENGHATLHGQSIDGFYAGSSIRYSCEVLHYLSGTETVTCTTNGTWSAPKPRCIKVITCQNPPVPSYGSVEIKPPSRTNSISRVGSPFLRLPRLPLPLARAAKPPPKPRSSQPSTVDLASKVKLPEGHYRVGSRAIYTCESRYYELLGSQGRRCDSNGNWSGRPASCIPVCGRSDSPRSPFIWNGNSTEIGQWPWQAGISRWLADHNMWFLQCGGSLLNEKWIVTAAHCVTYSATAEIIDPSQFKIYLGKYYRDDSRDDDYVQVREALEIHVNPNYDPGNLNFDIALIQLKTPVTLTTRVQPICLPTDITTREHLKEGTLAVVTGWGLNENNTYSEMIQQAVLPVVAASTCEEGYKEADLPLTVTENMFCAGYKKGRYDACSGDSGGPLVFADDSRTERRWVLEGIVSWGSPSGCGKANQYGGFTKVNVFLSWIRQFI

SEQ ID NO: 12 - protein, 1015 aa, Tachypleus tridentatus
MKFLVNVALVFMVVYISYIYARGVDLGLCDETRFECKCGDPGYVFNVPMKQCTYFYRWRPYCKPCDDLEAKDICPKYKRCQECKAGLDSCVTCPPNKYGTWCSGECQCKNGGICDQRTGACTCRDRYEGAHCEILKGCPLLPSDSQVQEVRNPPDNPQTIDYSCSPGFKLKGVARISCLPNGQWSSFPPKCIRECAKVSSPEHGKVNAPSGNMIEGATLRFSCDSPYYLIGQETLTCQGNGQWSGQIPQCKKLVFCPDLDPVNHAEHQVKIGVEQKYGQFPQGTEVTYTCSGNYFLMGFNTLKCNPDGSWSGSQPSCVKVADREVDCDSKAVDFLDDVGEPVRIHCPAGCSLTAGTVWGTAIYHELSSVCRAAIHAGKLPNSGGAVHVVNNGPYSDFLGSDLNGIKSEELKSLARSFRFDYVSSSTAGRSGCPDGWFEVEENCVYVTSKQRAWERAQGVCTNMAARLAVLDKDLIPSSLTETLRGKGLTTTWIGLHRLDAEKPFVWELMDRSNVVLNDNLTFWASGEPGNETNCVYLDIRDQLQPVWKTKSCFQPSSFACMMDLSDRNKAKCDDPGPLENGHATLHGQSIDGFYAGSSIRYSCEVLHYLSGTETVTCTTNGTWSAPKPRCIKVITCQNPPVPSYGSVEIKPPSRTNSISRVGSPFLRLPRLPLPLARAAKPPPKPRSSQPSTVDLASKVKLPEGHYRVGSRAIYTCESRYYELLGSQGRRCDSNGNWSGRPASCIPVCGRSDSPRSPFIWNGNSTEIGQWPWQAGISRWLADHNMWFLQCGGSLLNEKWIVTAAHCVTYSATAEIIDPSQFKIYLGKYYRDDSRDDDYVQVREALEIHVNPNYDPGNLNFDIALIQLKTPVTLTTRVQPICLPTDITTREHLKEGTLAVVTGWGLNENNTYSEMIQQAVLPVVAASTCEEGYKEADLPLTVTENMFCAGYKKGRYDACSGDSGGPLVFADDSRTERRWVLEGIVSWGSPSGCGKANQYGGFTKVNVFLSWIRQFI

SEQ ID NO: 13 - DNA, 1200 bp, Tachypleus tridentatus
atggacatgcgcgtgcctgcccaactgctcggcctgctgctgctgtggttcccgggtagccgctgtgtgggggtgctgttccccaagacccggaacgacaacgaatgcactgccaggggtggattgaaggggtcctgcaagagcctgatcgactgcccgtcggtgcttgccaccctgaaggactcctttcctgtcgtgtgctcctggaacggcagattccagccaattgtgtgctgtccggatgcaattgcccccccacctgtgaccaccaccgccgtgaccgtgatctctaccaaggaacccaagctgcctcggctccacatcagcggttgtgggaagcggaaggtcaagattgacattaccactgtgggacgctcaggatcaccgattctgcccccgatctccaccccgcaaaactccactggtggcaggggcatcattgccggtggagtggaagcgaagatcggagcctggccttggatggcggcggtgtttgtgaaaaacttcggaatcgggcggttccactgcgctggttcgatcatctccaacaagtacatcctgtccgccgcacatgccttcctgatcggaggccggaagttgacccccacgagactggccgtcagagtcggagggcattacatcaagcgcggacaggaataccccgtgaaggacgtgataatccacccccactacgtggaaaaggagaactacaatgacatcgccattatcgagctgaaggaggagctgaactttaccgatcttgtgaaccccatctgcctccctgaccccgagacagtgaccgacccactcaaagaccgcatcgtgactgcagctggatggggcgatctggacttcagcggaccgagaagccaggtcctgcgggaggtgtcaatcccggtggtgccagtcgacaaatgcgatcaggcctacgagaagctgaacactccctccctcaaaaacgggatcactaacaacttcctgtgcgcgggacttgaagaaggcggaaaggacgcctgtcagggcgattccggcggacctctgatgctcgtgaacaatactcggtggattgtcgtcggcgtggtgtccttcggacataagtgcgccgaggaaggctaccctggcgtctattcccgcgtggcctcctacctggactggattgccaaggtcaccaactcgctcgaccatgccgtgactaactga

SEQ ID NO: 14 - protein, 377 aa, Tachypleus tridentatus
VGVLFPKTRNDNECTARGGLKGSCKSLIDCPSVLATLKDSFPVVCSWNGRFQPIVCCPDAIAPPPVTTTAVTVISTKEPKLPRLHISGCGKRKVKIDITTVGRSGSPILPPISTPQNSTGGRGIIAGGVEAKIGAWPWMAAVFVKNFGIGRFHCAGSIISNKYILSAAHAFLIGGRKLTPTRLAVRVGGHYIKRGQEYPVKDVIIHPHYVEKENYNDIAIIELKEELNFTDLVNPICLPDPETVTDPLKDRIVTAAGWGDLDFSGPRSQVLREVSIPVVPVDKCDQAYEKLNTPSLKNGITNNFLCAGLEEGGKDACQGDSGGPLMLVNNTRWIVVGVVSFGHKCAEEGYPGVYSRVASYLDWIAKVTNSLDHAVTN

SEQ ID NO: 15 - protein, 399 aa, Tachypleus tridentatus
MDMRVPAQLLGLLLLWFPGSRCVGVLFPKTRNDNECTARGGLKGSCKSLIDCPSVLATLKDSFPVVCSWNGRFQPIVCCPDAIAPPPVTTTAVTVISTKEPKLPRLHISGCGKRKVKIDITTVGRSGSPILPPISTPQNSTGGRGIIAGGVEAKIGAWPWMAAVFVKNFGIGRFHCAGSIISNKYILSAAHAFLIGGRKLTPTRLAVRVGGHYIKRGQEYPVKDVIIHPHYVEKENYNDIAIIELKEELNFTDLVNPICLPDPETVTDPLKDRIVTAAGWGDLDFSGPRSQVLREVSIPVVPVDKCDQAYEKLNTPSLKNGITNNFLCAGLEEGGKDACQGDSGGPLMLVNNTRWIVVGVVSFGHKCAEEGYPGVYSRVASYLDWIAKVTNSLDHAVTN

SEQ ID NO: 16 - DNA, 1128 bp, Tachypleus tridentatus
atgctggtcaacaatgtcttttccctgctctgcttccctctcctgatgtccgtcgtgcgctgctccaccctctcgagacagcggcgccagtttgtgttccctgatgaagaggaactgtgttccaaccggttcaccgaagagggaacttgcaagaacgtgctggactgccgcatcctgcttcaaaagaacgactacaaccttctcaaggagtcaatctgcggtttcgaagggattaccccaaaagtctgctgcccgaagtcctcgcacgtgatctcaagcacccaggcaccccctgaaactaccactactgagcggcccccgaagcaaattcccccgaacttgccggaagtctgcggcatccacaacaccaccaccactaggatcatcggggggagagaagcccctatcggcgcctggccctggatgaccgctgtgtacattaagcagggcggtatccggagcgtgcagtgcggtggagctctggtcaccaatcgccacgtgatcacagcgtcacactgcgtcgtgaactcggcgggtaccgacgtgatgcccgccgacgtgttctctgtgcggttgggggaacataacctttactcgaccgatgatgactccaacccaatcgatttcgccgtgacgtccgtgaagcaccatgagcacttcgtgctggccacctacctgaacgacatagcaattctcactctgaacgacaccgtgacttttactgatcggatcaggccaatctgtctgccgtaccgcaagctcagatacgacgacctggccatgcgcaagccgttcattacgggttggggaactaccgccttcaacggaccttcctccgccgtgctgcgggaagtgcagctgccgatctgggagcacgaagcctgtagacaagcctacgaaaaggacctgaacattaccaacgtgtatatgtgtgccggattcgcggatggcggcaaagacgcatgccagggagactccggcggcccgatgatgctgcctgtgaaaaccggagagttctacctcatcggcatcgtcagcttcgggaagaaatgcgccctgcccggattccccggagtgtacactaaggtcaccgagttcctcgactggattgccgaacacatggtgtaa

SEQ ID NO: 17 - protein, 354 aa, Tachypleus tridentatus
STLSRQRRQFVFPDEEELCSNRFTEEGTCKNVLDCRILLQKNDYNLLKESICGFEGITPKVCCPKSSHVISSTQAPPETTTTERPPKQIPPNLPEVCGIHNTTTTRIIGGREAPIGAWPWMTAVYIKQGGIRSVQCGGALVTNRHVITASHCVVNSAGTDVMPADVFSVRLGEHNLYSTDDDSNPIDFAVTSVKHHEHFVLATYLNDIAILTLNDTVTFTDRIRPICLPYRKLRYDDLAMRKPFITGWGTTAFNGPSSAVLREVQLPIWEHEACRQAYEKDLNITNVYMCAGFADGGKDACQGDSGGPMMLPVKTGEFYLIGIVSFGKKCALPGFPGVYTKVTEFLDWIAEHMV

SEQ ID NO: 18 - protein, 375 aa, Tachypleus tridentatus
MLVNNVFSLLCFPLLMSVVRCSTLSRQRRQFVFPDEEELCSNRFTEEGTCKNVLDCRILLQKNDYNLLKESICGFEGITPKVCCPKSSHVISSTQAPPETTTTERPPKQIPPNLPEVCGIHNTTTTRIIGGREAPIGAWPWMTAVYIKQGGIRSVQCGGALVTNRHVITASHCVVNSAGTDVMPADVFSVRLGEHNLYSTDDDSNPIDFAVTSVKHHEHFVLATYLNDIAILTLNDTVTFTDRIRPICLPYRKLRYDDLAMRKPFITGWGTTAFNGPSSAVLREVQLPIWEHEACRQAYEKDLNITNVYMCAGFADGGKDACQGDSGGPMMLPVKTGEFYLIGIVSFGKKCALPGFPGVYTKVTEFLDWIAEHMV

SEQ ID NO: 19 - protein, 4 aa, Artificial Sequence
  Description of Artificial Sequence Synthetic peptide
IEGR

(19) United States
(12) Patent Application Publication
(10) Pub. No.: US 2024/0125789 A1
(43) Pub. Date: Apr. 18, 2024

(54) HYBRID AMEBOCYTE LYSATE AND USES THEREOF

(71) Applicant: Charles River Laboratories, Inc.

(72) Inventors: Norman R. Wainwright, Skaneateles, NY (US); Masakazu Tsuchiya, Mount Pleasant, SC (US); John Dubczak, Summerville, SC (US); Foster T. Jordan, Chapin, SC (US)

(73) Assignee: Charles River Laboratories, Inc., Wilmington, MA (US)

(21) Appl. No.: 18/275,777

(22) PCT Filed: Feb. 11, 2022
(86) PCT No.: PCT/US2022/016185

§ 371 Date: Aug. 3, 2023

Related U.S. Application Data
(60) Provisional application No. 63/148,916, filed on Feb. 12, 2021.

(51) Int. Cl. G01N 33/579; C12N 9/64

(52) U.S. Cl. CPC G01N 33/579; C12N 9/6408; C12Y 304/21084; C12Y 304/21085; C12Y 304/21086; G01N 2333/96411

(57) ABSTRACT

The invention relates generally to hybrid amebocyte lysate compositions (including both native and recombinant components) and their use in detecting and/or quantifying endotoxin in a sample.

### CROSS-REFERENCE TO RELATED APPLICATIONS

This application claims the benefit of and priority to U.S. Provisional Patent Application No. 63/148,916, filed Feb. 12, 2021, the disclosure of which is incorporated herein by reference in its entirety for all purposes.

### FIELD OF THE INVENTION

The present invention relates generally to methods and compositions for detecting and/or quantifying endotoxin in a sample. More particularly, the invention relates to hybrid amebocyte lysates (including native and recombinantly produced components) and their use for detecting and/or quantifying endotoxin in a sample.

### BACKGROUND

Microbial contamination by, for example. Gram negative bacteria, may cause severe illness and, in some cases, even death in humans. Manufacturers in certain industries, for example, the pharmaceutical, medical device, and food industries, must meet exacting standards to verity that their products do not contain levels of microbial contaminants that would otherwise compromise the health of the recipient. These industries require frequent, accurate, and sensitive testing for the presence of such microbial contaminants to meet certain standards, for example, standards imposed by the United States Food and Drug Administration (USFDA) or Environmental Protection Agency. By way of example, the USFDA requires certain manufacturers of pharmaceuticals and invasive medical devices to establish that their products are free of detectable levels of Gram negative bacterial endotoxin.

Furthermore, when people become infected with Gram negative bacteria, the bacteria may produce and secrete fever-inducing bacterial endotoxins. Bacterial endotoxins can be dangerous and even deadly to humans. Symptoms of infection may range from fever, in mild cases, to death. In order to promptly initiate proper medical treatment, it usually is important to identify, as early as possible, the presence of an endotoxin and, if possible, the concentration of the endotoxin in the patient.

To date, a variety of assays have been developed to detect the presence and/or amount of endotoxin in a test sample. One family of assays use hemocyte lysates prepared from the hemolymph of crustaceans, for example, horseshoe crabs. These assays typically exploit, in one way or another, a clotting cascade that occurs when the hemocyte lysate is exposed to endotoxin. A currently preferred hemocyte lysate is amebocyte lysate (AL) produced from the hemolymph of a horseshoe crab, for example, Limulus polyphemus, Tachypleus tridentatus, Tachypleus gigas, and Carcinoscorpius rotundicauda.

These assays use blood that is harvested from horseshoe crabs, which has resulted in concerns over the ecological sustainability of this practice. However, to date, fully synthetic or recombinant amebocyte lysate reagents are not comparable in performance to native amebocyte lysate (Dubczak et al. (2021) EUR. J. PHRM. SCI. 159:105716). Accordingly, there exists a need for new reagents that reduce the burden on the horseshoe crab population while adequately detecting endotoxin at the same level of sensitivity and accuracy as naturally derived amebocyte lysates.

### SUMMARY OF THE INVENTION

The invention is based, in part, upon the discovery that the addition of a recombinant factor B and/or a recombinant proclotting enzyme to a native amebocyte lysate, e.g., for example, a diluted native amebocyte lysate, to produce a hybrid amebocyte lysate can reduce the amount of the native amebocyte lysate required to detect endotoxin in a sample, while maintaining, or in certain instances even exceeding the sensitivity and/or activity of the native amebocyte lysate for endotoxin. Furthermore, it has been discovered that hybrid amebocyte lysates described herein are superior to fully recombinant reagents, and comparable or superior to native amebocyte lysates, at detecting naturally occurring endotoxins.

Accordingly, in one aspect, the invention provides a hybrid amebocyte lysate composition. The composition comprises a native horseshoe crab amebocyte lysate. The composition also comprises a recombinant horseshoe crab factor B and/or a recombinant horseshoe crab proclotting enzyme.

In another aspect, the invention provides a hybrid amebocyte lysate composition. The composition comprises a native horseshoe crab factor C, a native horseshoe crab factor B, and a native horseshoe crab pro-clotting enzyme. The composition also comprises a recombinant horseshoe crab factor B and/or a recombinant horseshoe crab proclotting enzyme.

In another aspect, the invention provides a hybrid amebocyte lysate composition. The composition comprises a horseshoe crab factor C, horseshoe crab factor B, and a horseshoe crab proclotting enzyme, wherein (a) the ratio of horseshoe crab factor B to horseshoe crab factor C is greater than the ratio of horseshoe crab factor B to horseshoe crab factor C in a native horseshoe crab amebocyte lysate, and/or (b) the ratio of horseshoe crab proclotting enzyme to horseshoe crab factor C is greater than the ratio of horseshoe crab proclotting enzyme to horseshoe crab factor C in a native horseshoe crab amebocyte lysate.

In certain embodiments of any of the foregoing hybrid amebocyte lysate compositions, the composition does not comprise a recombinant horseshoe crab factor C.

In certain embodiments of any of the foregoing hybrid amebocyte lysate compositions, the composition comprises from about 0.05 to about 1 U/mL for example, from about 0.1 to about 0.5 U/mL, of recombinant horseshoe crab factor B.

In certain embodiments of any of the foregoing hybrid amebocyte lysate compositions, the composition comprises from 0.05 to about 2,000 U/mL, for example, from about 50 to about 200 U/mL, of recombinant proclotting enzyme.

In certain embodiments of any of the foregoing hybrid amebocyte lysate compositions, the composition has substantially the same or greater sensitivity in detecting endotoxin than native horseshoe crab amebocyte lysate. In certain embodiments, the composition retains substantially the same activity when stored at 4° C. for 3, 4, 5, 6, 7, or 8 hours.

In certain embodiments of any of the foregoing hybrid amebocyte lysate compositions, the horseshoe crab amebocyte lysate is a Limulus polyphemus amebocyte lysate. In certain embodiments, the horseshoe crab factor B is a Limulus polyphemus factor B. In certain embodiments, the horseshoe crab proclotting enzyme is a Limulus polyphemus proclotting enzyme. In certain embodiments, the horseshoe crab factor C is a Limulus polyphemus factor C.

In certain embodiments of any of the foregoing hybrid amebocyte lysate compositions, the horseshoe crab amebocyte lysate is a Tachypleus amebocyte lysate. In certain embodiments, the horseshoe crab factor B is a Tachypleus factor B. In certain embodiments, the horseshoe crab proclotting enzyme is a Tachypleus proclotting enzyme. In certain embodiments, the horseshoe crab factor C is a Tachypleus factor C.

In certain embodiments of any of the foregoing hybrid amebocyte lysate compositions, the recombinant horseshoe crab factor B and/or the recombinant horseshoe crab proclotting enzyme are expressed in a mammalian cell, for example, a Chinese hamster ovary (CHO) or human embryonic kidney (HEK) cell. In certain embodiments, the recombinant factor B and/or the recombinant proclotting enzyme has different glycosylation than the factor B and/or the proclotting enzyme present in the native horseshoe crab amebocyte lysate.

In another aspect, the invention provides a method for preparing an endotoxin detection reagent. The method comprises adding a recombinant horseshoe crab factor B and/or a recombinant horseshoe crab proclotting enzyme to a native horseshoe crab amebocyte lysate.

In another aspect, the invention provides a method for increasing the endotoxin sensitivity of a native horseshoe crab amebocyte lysate. The method comprises adding to the native horseshoe crab amebocyte lysate a recombinant horseshoe crab factor B and/or a recombinant horseshoe crab proclotting enzyme, thereby to increase the endotoxin sensitivity of the native horseshoe crab amebocyte lysate.

In another aspect, the invention provides a method for reducing the amount of a native horseshoe crab amebocyte lysate required to detect endotoxin. The method comprises: (i) diluting the native horseshoe crab amebocyte lysate; and (ii) adding to the diluted native horseshoe crab amebocyte lysate a recombinant horseshoe crab factor B and/or a recombinant horseshoe crab proclotting enzyme to produce a hybrid amebocyte lysate.

In certain embodiments of any of the foregoing methods, the method does not comprise adding a recombinant horseshoe crab factor C to the native horseshoe crab amebocyte lysate or diluted native horseshoe crab amebocyte lysate.

In certain embodiments of any of the foregoing methods, the method comprises adding from about 0.05 to about 1 U/mL, e.g., from about 0.1 to about 0.5 U/mL, of recombinant horseshoe crab factor B to the native horseshoe crab amebocyte lysate or diluted native horseshoe crab amebocyte lysate.

In certain embodiments of any of the foregoing methods, the method comprises adding from 0.05 to about 2,000 U/mL, e.g., from about 50 to about 200 U/mL, of recombinant proclotting enzyme to the native horseshoe crab amebocyte lysate or diluted native horseshoe crab amebocyte lysate.

In certain embodiments of any of the foregoing methods, the method results in a composition that has substantially the same or greater sensitivity in detecting endotoxin than native horseshoe crab amebocyte lysate.

In certain embodiments of any of the foregoing methods, the horseshoe crab amebocyte lysate is a Limulus polyphemus amebocyte lysate. In certain embodiments, the horseshoe crab factor B is a Limulus polyphemus factor B. In certain embodiments, the horseshoe crab proclotting enzyme is a Limulus polyphemus proclotting enzyme. In certain embodiments, the horseshoe crab factor C is a Limulus polyphemus factor C.

In certain embodiments of any of the foregoing methods, the recombinant horseshoe crab factor B and/or the recombinant horseshoe crab proclotting enzyme are expressed in a mammalian cell, for example, a Chinese hamster ovary (CHO) or human embryonic kidney (HEK) cell. In certain embodiments, the recombinant factor B and/or the recombinant proclotting enzyme has different glycosylation than the factor B and/or the proclotting enzyme present in the native horseshoe crab amebocyte lysate.

These and other aspects and features of the invention are described in the following detailed description and claims.

### DESCRIPTION OF THE DRAWINGS

The invention can be more completely understood with reference to the following drawings.

FIG. 1 is a schematic representation of the coagulation system present in amebocytes.

FIG. 2 is bar graph depicting relative activity of the indicated LAL reagents in a kinetic chromogenic assay (KCA).

FIG. 3 is bar graph depicting relative activity of the indicated LAL reagents in a kinetic chromogenic assay (KCA).

FIG. 4 is a line graph depicting onset time for the indicated LAL reagents as a function of endotoxin concentration in a kinetic chromogenic assay (KCA).

FIG. 5A is a line graph depicting onset time for a hybrid LAL reagent as a function of endotoxin concentration in a kinetic turbidometric assay (KTA). FIG. 5B is a chart depicting gel clot formation using a hybrid LAL reagent at the indicated endotoxin concentrations.

FIG. 6A is a line graph depicting onset time for a hybrid LAL reagent as a function of endotoxin concentration in a kinetic turbidometric assay (KTA). FIG. 6B is a chart depicting gel clot formation using a hybrid LAL reagent at the indicated endotoxin concentrations.

FIG. 7 is a line graph depicting onset time for the indicated TAL reagents as a function of endotoxin concentration in a kinetic chromogenic assay (KCA).

FIGS. 8A-8D are schematic illustrations in perspective view (FIG. 8A), top view (FIG. 8B), side view (FIG. 8C), and end view (FIG. 8D), of an exemplary cartridge useful in performing assays with the hybrid lysate described herein.

FIG. 9 is a line graph depicting onset time for the indicated reagents as a function of endotoxin concentration in a kinetic chromogenic assay (KCA).

FIG. 10 is a line graph depicting onset time for the indicated reagents as a function of endotoxin concentration in a kinetic chromogenic assay (KCA).

### DETAILED DESCRIPTION

The invention is based, in part, upon the discovery that addition of a recombinant factor B and/or a recombinant proclotting enzyme to a native amebocyte lysate to produce a hybrid amebocyte lysate can reduce the amount of the native amebocyte lysate required to detect endotoxin in a sample, while maintaining, or in certain instances even increasing the sensitivity and/or activity of the amebocyte lysate. Furthermore, it has been discovered that hybrid amebocyte lysates described herein are superior to fully recombinant reagents, and comparable or superior to native amebocyte lysates, at detecting naturally occurring endotoxins.

Various features and aspects of the invention are discussed in more detail below.

### I. Hybrid Amebocyte Lysate

The invention relates, in part, to hybrid amebocyte lysate compositions including one or more native components (e.g., a native horseshoe crab amebocyte lysate, a native horseshoe crab factor C, a native horseshoe crab factor B, and/or a native horseshoe crab pro-clotting enzyme) and one or more recombinant components (e.g., a recombinant horseshoe crab factor B, a recombinant horseshoe crab proclotting enzyme, and/or a recombinant horseshoe crab factor C).

In one aspect, the invention provides a hybrid amebocyte lysate composition comprising: (i) a native horseshoe crab amebocyte lysate, and (ii) a recombinant horseshoe crab factor B and/or a recombinant horseshoe crab proclotting enzyme.

In one aspect, the invention provides a hybrid amebocyte lysate composition comprising: (i) a native horseshoe crab factor C, (ii) a native horseshoe crab factor B, (iii) a native horseshoe crab pro-clotting enzyme, and (iv) a recombinant horseshoe crab factor B and/or a recombinant horseshoe crab proclotting enzyme.

In one aspect, the invention provides an amebocyte lysate composition comprising: (i) a horseshoe crab factor C, (ii) horseshoe crab factor B, and (iii) a horseshoe crab proclotting enzyme, wherein (a) the ratio of horseshoe crab factor B to horseshoe crab factor C is greater than the ratio of horseshoe crab factor B to horseshoe crab factor C in a native horseshoe crab amebocyte lysate, and/or (b) the ratio of horseshoe crab proclotting enzyme to horseshoe crab factor C is greater than the ratio of horseshoe crab proclotting enzyme to horseshoe crab factor C in a native horseshoe crab amebocyte lysate. It is understood that the ratio of horseshoe crab factor B to horseshoe crab factor C or the ratio of horseshoe crab proclotting enzyme to horseshoe crab factor C may refer to a ratio of enzymatic activity. Enzymatic activity may be measured by an method known in the art (e.g., as described in Example 1 hereinbelow). Alternatively, the ratio of horseshoe crab factor B to horseshoe crab factor C or the ratio of horseshoe crab proclotting enzyme to horseshoe crab factor C may refer to a ratio of protein quantity. Protein quantity may be measured by any method known in the art, including for example, Western blot, ELISA, or ultraviolet (UV) absorption at 280 nm.

In certain embodiments, a hybrid amebocyte lysate composition does not contain recombinant horseshoe crab factor C.

In certain embodiments, a hybrid amebocyte lysate composition has substantially the same or greater sensitivity for a microbial contaminant (e.g., endotoxin) as a corresponding native horseshoe crab amebocyte lysate. For example, the hybrid amebocyte lysate composition may have at least 50%, at least 60%, at least 70%, at least 80%, at least 90%, at least 95%, at least 96%, at least 97%, at least 98%, at least 99%, at least 100%, at least 110%, at least 120%, at least 130%, at least 140%, at least 150%, at least 175%, at least 200%, at least 225%, at least 250%, at least 300%, at least 350%, or at least 400% of the sensitivity of the corresponding native horseshoe crab amebocyte lysate. Amebocyte lysate sensitivity may be assayed by any method known in the art, including, for example, a kinetic chromogenic assay method (KCA), as described in Example 2 hereinbelow.

In certain embodiments, a hybrid amebocyte lysate composition containing less crude lysate than a reference native amebocyte lysate e.g., containing 10%, 20%, 30%, 40%, 50%, 60%, 70%, 80%, or 90% of the crude lysate of a reference native amebocyte lysate) has the substantially the same or greater sensitivity for a microbial contaminant (e.g., endotoxin) as the reference native horseshoe crab amebocyte lysate. For example, the hybrid amebocyte lysate composition may have at least 50%, at least 60%, at least 70%, at least 80%, at least 90%, at least 95%, at least 96%, at least 97%, at least 98%, at least 99%, at least 100%, at least 110%, at least 120%, at least 130%, at least 140%, at least 150%, at least 175%, at least 200%, at least 225%, at least 250%, at least 300%, at least 350%, or at least 400% of the sensitivity of the reference native horseshoe crab amebocyte lysate. In certain embodiments, the hybrid amebocyte lysate composition is substantially the same as the reference native amebocyte lysate but for (i) the amount of crude lysate present, and (ii) the presence or absence of any recombinant proteins. Amebocyte lysate sensitivity may be assayed by any method known in the art, including, for example, a kinetic chromogenic assay method (KCA), as described in Example 2 hereinbelow.

In certain embodiments, a hybrid amebocyte lysate composition retains substantially the same activity when stored at 4° C. for at least 3, 4, 5, 6, 7, 8, 9, 10, 11, or 12 hours, or 3, 4, 5, 6, 7, or S hours.

### A. Native Horseshoe Crab Amebocyte Lysate

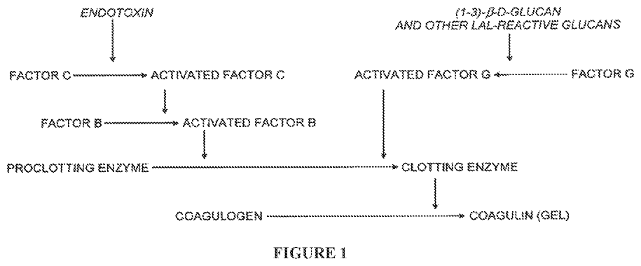

As shown in FIG. 1, the coagulation system of hemolymph, like the mammalian blood coagulation system, comprises at least two coagulation cascades that include an endotoxin-mediated pathway (the Factor C pathway) and a (1→3)-B-D glucan-mediated pathway (the Factor G pathway).

When bacterial endotoxin is contacted with LAL, the endotoxin initiates a series of enzymatic reactions, referred to in the art as the Factor C pathway, that can involve three serine protease zymogens called Factor C, Factor B, and pro-clotting enzyme (see, FIG. 1). Briefly, upon exposure to endotoxin, the endotoxin-sensitive factor, Factor C, is activated. Activated Factor C thereafter hydrolyses and activates Factor B, whereupon activated Factor B activates proclotting enzyme to produce clotting enzyme. The clotting enzyme thereafter hydrolyzes specific sites, for example, Arg¹⁸-Thr¹⁹ and Arg⁴⁶-Gly⁴⁷ of coagulogen, an invertebrate, fibrinogen-like clottable protein, to produce a coagulin gel. See, for example, U.S. Pat. No. 5,605,806.

(1→3)-B-D glucans and other amebocyte lysate reactive Oilcans, produced by microorganisms such as yeasts and molds, can also activate the clotting cascade of amebocyte lysates, through a different enzymatic pathway, referred to in the art as the Factor G pathway (see, FIG. 1). Factor G is a serine protease zymogen that becomes activated by (1→3)-β-D glucan or other LAL reactive glucans. Upon exposure to (1→3)-β-D glucan, for example. Factor G is activated to produce activated Factor G. Activated Factor G thereafter converts the proclotting enzyme into clotting enzyme, whereupon the clotting enzyme converts coagulogen into coagulin.

As used herein, the term “native horseshoe crab amebocyte lysate” is understood to mean any lysate or fraction thereof (e.g., the components of a factor C mediated cascade) produced by the lysis, extrusion, or extraction of the cellular contents from amebocytes extracted from a horseshoe crab. Under certain circumstances, the native amebocyte lysate comprises the naturally occurring components of an enzymatic cascade as shown in FIG. 1) produced, for example, by the lysis, extrusion, or extraction of the cellular contents from amebocytes extracted from a horseshoe crab. A native horse shoe crab amebocyte lysate, which does not contain a recombinant zymogen or functional fragment thereof (e.g., recombinant factor B, recombinant factor C or recombinant proclotting enzyme) can be used to produce a hybrid lysate described herein, by supplementation of the native amebocyte lysate with, for example, a recombinant zymogen or functional fragment thereof (e.g., recombinant factor B, recombinant factor C or recombinant proclotting enzyme). Depending upon the components present in, or admixed with, the lysate it may produce a clot in the presence of an endotoxin, for example, a Gram negative bacterial endotoxin and/or a glucan, for example, a (1→3)β-D glucan, produced by a yeast or a mold. Preferred amebocyte lysates can be derived from horseshoe crabs belonging to the Limulus genus, for example, Limulus polyphemus, the Tachypleus genus, for example, Tachypleus tridentatus and Tachypleus gigas, and the Carcinoscorpius genus, for example, Carcinoscorpius rotundicauda.

In certain embodiments, a native horseshoe crab amebocyte lysate includes each of a native factor C, a native factor B, and a native proclotting enzyme.

### B. Methods of Makin Native Horseshoe Crab Amebocyte Lysate

Crude lysates may be produced using the procedure described in Levin et al. (1968) THROMB. DIATH. HAEMORRH. 19: 186, with modification, or in Prior 1990 “Clinical Applications of the Limulus Amebocyte Lysate Test” CRC PRESS 28-36 and 159-166, and in U.S. Pat. No. 4,322,217. Other lysates may include those, for example, described in U.S. Pat. Nos. 6,270,982 and 6,391,570. In certain embodiments, a crude lysate is produced as described in Example 1 hereinbelow.

It is possible to produce an endotoxin-specific lysate by removing Factor G activity from the lysate, such as the Factor G depleted lysates produced by the methods described in U.S. Pat. Nos. 6,391,570 and 6,270,982. Also, it is contemplated that lysates may be depleted of Factor G activity by the addition of certain inhibitors or modulators of Factor G activity, for example, certain detergents, saccharides, polysaccharides, and other reagents described in U.S. Pat. Nos. 5,155,032; 5,179,006; 5,318,893; 5,474,984; 5,641,643; and 6,270,982. An endotoxin-specific lysate is a lysate capable of reacting with a bacterial endotoxin but in which the reactivity to (1→3)-B-D glucan has been depleted by at least 80%, more preferably by at least 90%, and more preferably by at least 95% relative to the crude lysate from which the endotoxin-specific lysate was prepared.

Methods for enhancing the sensitivity of hemocyte lysate for endotoxin, for example, may include, without limitation, aging the crude hemocyte lysate, adjusting pH, adjusting the concentration of divalent cations, adjusting the concentration of coagulogen, chloroform extraction, and the addition of serum albumin, biocompatible buffers and/or biological detergents.

As will be apparent to one of ordinary skill, divalent metal salts, which are known to promote activation of the pro-clotting enzyme of hemocyte lysate, as well as buffers to avoid extremes of pH that could inactivate the clotting enzyme preferably are included in the lysate. Any of the buffers and salts that are understood in the art to be compatible with the amebocyte lysate system may be used. Typical formulation additives may include, without limitation, about 100-300 mM NaCl, about 10-100 mM divalent cations (e.g., Mg²⁺), biocompatible buffers, e.g., Tris(tris(hydroxy)aminomethane), to give a final pH of about 6.0 to about 8.0, and, if the lysate is to be freeze dried, then sugars, e.g., mannitol or dextran. It is contemplated that the choice of appropriate formulation additives may also be determined by routine experimentation.

### C. Factor B

As used herein, the term “factor B” refers to a zymogen, or a functional fragment thereof that is capable of being activated upon cleavage by a factor C, and is capable of cleaving (e.g., enzymatically cleaving) a proclotting enzyme to form an active clotting enzyme. The term factor B includes variants having one or more amino acid substitutions, deletions, or insertions relative to a wild-type factor B sequence, and/or fusion proteins or conjugates including a factor B. As used herein, the term “functional fragment” of a factor B refers to fragment of a full-length factor B that retains, for example, at least 10%, at least 20%, at least 30%, at least 40%, at least 50%, at least 60%, at least 70%, at least 80%, at least 90%, at least 95%, at least 96%, at least 97%, at least 98%, at least 99%, or 100% of the enzymatic activity of the corresponding full-length, naturally occurring factor B. Factor B enzymatic activity may be assayed by any method known in the art, including, for example, by measuring cleavage of the chromogenic substrate H-D-Leu-Thr-Arg-pNA, for example, as described in Example 1 hereinbelow. In certain embodiments, the functional fragment comprises at least 100, 150, 200, 250, 300, 350, 360, 370, 380, or 390 consecutive amino acids present in a full-length, naturally occurring factor B.

It is contemplated that any horseshoe crab factor B, for example, a Limulus polyphemus, Tachypleus tridentatus, Tachypleus gigas, or Carcinoscorpius rotundicauda factor B, may be used in the practice of the invention.

A DNA sequence encoding an exemplary Limulus polyphemus factor B is depicted in SEQ ID NO: 4. Exemplary Limulus polyphemus factor B amino acid sequences are depicted in SEQ ID NOs: 5 and 6. SEQ ID NO: 5 is the mature form whereas SEQ ID NO: 6 includes the signal sequence as residues 1-25. A DNA sequence encoding an exemplary Tachypleus tridentatus factor B is depicted in SEQ D NO: 13. Exemplary Tachypleus tridentatus factor B amino acid sequences are depicted in SEQ ID NOs: 14 and 15. SEQ ID NO: 14 is the mature form whereas SEQ ID NO: 15 includes the signal sequence as residues 1-22. In certain embodiments, a factor B comprises the amino acid sequence of SEQ ID NO: 5, SEQ ID NO: 6, SEQ ID NO: 14, or SEQ ID NO: 15 or an amino acid sequence having at least 70%, at least 80%, at least 90%, at least 95%, at least 96%, at least 97%, at least 98%, at least 99%, at least 99.5%, or at least 99.8% identity to the amino acid sequence of SEQ ID NO: 5, SEQ II) NO: 6, SEQ ID NO: 14, or SEQ ID NO: 15.

Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al. (1990) PROC. NATL. ACAD. SCI. USA 87:2264-2268, Altschul. (1993) J. MOL. EVOL. 36, 290-300; Altschul et al. (1997) NUCLEIC ACIDS RES. 25:3389-3402, incorporated by reference) are tailored for sequence similarity searching. For a discussion of basic issues in searching sequence databases, see Altschul et al, (1994) NATURE GENETICS 6:119-129, which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al. (1992) PROC. NATL. ACAD. SCI. USA 89:10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: −G, Cost to open gap [Integer]: default=5 for nucleotides/11 for proteins; −E, Cost to extend gap [Integer]: default=2 for nucleotides/1 for proteins; −q, Penalty for nucleotide mismatch [Integer]: default=−3; −r, reward for nucleotide match [Integer]: default=1; expect value [Real]: default=10; −W, wordsize [Integer]: default=11 for nucleotides/28 for megablast/3 for proteins; −y, Dropoff (X) for blast extensions in bits: default=20 for blastn/7 for others; −X, X dropoff value for gapped alignment (in bits): default=15 for all programs, not applicable to blastn; and −Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

In certain embodiments, the factor B comprises a conservative substitution relative to a wild-type factor B sequence or a factor B sequence disclosed herein. As used herein, the term “conservative substitution” refers to a substitution with a structurally similar amino acid. For example, conservative substitutions may include those within the following groups: Ser and Cys; Leu, Ile, and Val; Glu and Asp; Gln and Asn; Lys, Arg and His; Phe, Tyr, and Trp. Conservative substitutions may also be defined by the BLAST (Basic Local Alignment Search Tool) algorithm, the BLOSUM substitution matrix (e.g., BLOSUM 62 matrix), or the PAM substitution:p matrix (e.g., the PAM 250 matrix).

In certain embodiments, the factor B is a recombinant factor B. As used herein, when referring to a protein or polypeptide, the term “recombinant” refers to a protein or polypeptide which is produced by recombinant nucleic acid, e.g. recombinant DNA, techniques, wherein generally, DNA or other nucleic acid encoding the protein or polypeptide is inserted into a suitable expression vector which is in turn introduced in to a host cell to produce the heterologous protein within the host cell.

In certain embodiments, the factor B is a native factor B. As used herein, when referring to a protein or polypeptide (e.g., native factor B), the “native” refers to a protein or polypeptide derived from a natural source as opposed to a protein or polypeptide produced by using recombinant means, e.g., recombinant DNA technologies. A native protein or polypeptide (e.g., factor B) may be isolated. As used herein, term “isolated” refers to molecules or biological or cellular materials that are in an environment that is different from their naturally occurring environment. The term “isolated” encompasses a nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in a natural source.

A contemplated composition may comprise, for example, from about 0.01 to about 5 U/mL, from about 0.01 to about 3 U/mL, from about 0.01 to about 1 U/mL, from about 0.01 to about 0.9 U/mL from about 0.01 to about 0.8 U/mL, from about 0.01 to about 0.7 U/mL, from about 0.01 to about 0.6 U/mL, from about 0.01 to about 0.5 U/mL, from about 0.01 to about 0.4 U/mL, from about 0.01 to about 0.3 U/mL, from about 0.01 to about 0.2 U/mL, from about 0.01 to about 0.1 U/mL, from about 0.01 to about 0.075 U/mL, from about 0.01 to about 0.05 U/mL, from about 0.05 to about 5 U/mL, from about 0.05 to about 3 U/mL, from about 0.05 to about 1 U/mL, from about 0.05 to about 0.9 U/mL, from about 0.05 to about 0.8 U/mL, from about 0.05 to about 0.7 U/mL, from about 0.05 to about 0.6 U/mL, from about 0.05 to about 0.5 U/mL, from about 0.05 to about 0.4 U/mL, from about 0.05 to about 0.3 U/mL, from about 0.05 to about 0.2 U/mL, from about 0.05 to about 0.1 U/mL, from about 0.05 to about 0.075 U/mL, from about 0.075 to about 5 U/mL, from about 0.075 to about 3 U/mL, from about 0.075 to about 1 U/mL, from about 0.075 to about 0.9 U/mL, from about 0.075 to about 0.8 U/mL, from about 0.075 to about 0.7 U/mL, from about 0.075 to about 0.6 U/mL, from about 0.075 to about 0.5 U/mL, from about 0.075 to about 0.4 U/mL, from about 0.075 to about 0.3 U/mL, from about 0.075 to about 0.2 U/mL, from about 0.075 to about 0.1 U/mL, from about 0.1 to about 5 U/mL, from about 0.1 to about 3 U/mL, from about 0.1 to about 1 U/mL, from about 0.1 to about 0.9 U/mL, from about 0.1 to about 0.8 U/mL, from about 0.1 to about 0.7 U/mL, from about 0.1 to about 0.6 from about 0.1 to about 0.5 U/mL, from about 0.1 to about 0.4 U/mL, from about 0.1 to about 0.3 U/mL, from about 0.1 to about 0.2 U/mL, from about 0.2 to about 5 U/mL, from about 0.2 to about 3 U/mL, from about 0.2 to about 1 U/mL, from about 0.2 to about 0.9 U/mL, from about 0.2 to about 0.8 U/mL, from about 0.2 to about 0.7 U/mL, from about 0.2 to about 0.6 U/mL, from about 0.2 to about 0.5 U/mL, from about 0.2 to about 0.4 U/mL, from about 0.2 to about 0.3 from about 0.3 to about 5 U/mL, from about 0.3 to about 3 U/mL, from about 0.3 to about 1 U/mL, from about 0.3 to about 0.9 U/mL, from about 0.3 to about 0.8 U/mL, from about 0.3 to about 0.7 U/mL, from about 0.3 to about 0.6 U/mL, from about 0.3 to about 0.5 U/mL, from about 0.3 to about 0.4 U/mL, from about 0.4 to about 5 U/mL, from about 0.4 to about 3 U/mL, from about 0.4 to about 1 U/mL, from about 0.4 to about 0.9 U/mL, from about 0.4 to about 0.8 U/mL, from about 0.4 to about 0.7 U/mL, from about 0.4 to about 0.6 U/mL, from about 0.4 to about 0.5 U/mL, from about 0.5 to about 5 U/mL, from about 0.5 to about 3 U/mL, from about 0.5 to about 1 U/mL, from about 0.5 to about 0.9 U/mL, from about 0.5 to about 0.8 U/mL, from about 0.5 to about 0.7 U/mL, from about 0.5 to about 0.6 U/mL, from about 0.6 to about 5 U/mL, from about 0.6 to about 3 U/mL, from about 0.6 to about 1 U/mL, from about 0.6 to about 0.9 U/mL, from about 0.6 to about 0.8 U/mL, from about 0.6 to about 0.7 U/mL, from about 0.7 to about 5 U/mL, from about 0.7 to about 3 U/mL, from about 0.7 to about 1 U/mL, from about 0.7 to about 0.9 U/mL, from about 0.7 to about 0.8 U/mL, from about 0.8 to about 5 U/mL, from about 0.8 to about 3 U/mL, from about 0.8 to about 1 U/mL, from about 0.8 to about 0.9 U/mL, from about 0.9 to about 5 U/mL, from about 0.9 to about 3 U/mL, from about 0.9 to about 1 U/mL, from about 1 to about 5 U/mL, from about 1 to about 3 U/mL, or from about 3 to about 5 U/mL of factor B (e.g., recombinant factor B). Factor B enzymatic activity may be assayed by any method known in the art, including, for example, by measuring cleavage of the chromogenic substrate H-D-Leu-Thr-Arg-pNA, for example, as described in Example 1 herein. As used herein one unit (U) of factor B activity is defined as the amount of enzyme that catalyzes the conversion of 1 micromole of a substrate (e.g., H-D-Leu-Thr-Arg-pNA) per minute.

### D. Proclotting Enzyme

As used herein, the term “proclotting enzyme” refers to a zymogen, or a functional fragment thereof, that is capable of being activated upon cleavage by an activated factor B, and is capable of cleaving (e.g., enzymatically cleaving) coagulogen to form coagulin. The term proclotting enzyme includes variants having one or more amino acid substitutions, deletions, or insertions relative to a wild-type proclotting enzyme sequence, and/or fusion proteins or conjugates including a proclotting enzyme. As used herein, the term “functional fragment” of a proclotting enzyme refers to fragment of a full-length proclotting enzyme that retains, for example, at least 10%, at least 20%, at least 30%, at least 40%, at least 50%, at least 60%, at least 70%, at least 80%, at least 90%, at least 95%, at least 96%, at least 97%, at least 98%, at least 99%, or 100% of the enzymatic activity of the corresponding full-length, naturally occurring proclotting enzyme. Proclotting enzyme enzymatic activity may be assayed by any method known in the art, including, for example, by measuring cleavage of the chromogenic substrate Ac-Ilu-Glu-Gly-Arg-pNA, for example, as described in Example 1 herein. In certain embodiments, the functional fragment comprises at least 100, 150, 200, 250, 300, 320, 330, 340, 350, 360, or 370 consecutive amino acids present in chill-length, naturally occurring proclotting enzyme.

It is contemplated that any horseshoe crab factor proclotting enzyme, for example, a Limulus polyphemus, Tachypleus tridentatus, Tachypleus gigas, or Carcinoscorpius rotundicauda proclotting enzyme, may be used in the practice of the invention.

A DNA sequence encoding an exemplary Limulus polyphemus proclotting enzyme is depicted in SEQ ID NO: 7. Exemplary Limulus polyphemus proclotting enzyme amino acid sequences are depicted in SEQ ID NOs: 8 and 9. SEQ ID NO: 8 is the mature form whereas SEQ ID NO:9 includes the signal sequence as residues 1-28. A DNA sequence encoding an exemplary Tachypleus tridentatus proclotting enzyme is depicted in SEQ ID NO: 16. Exemplary Tachypleus tridentatus proclotting enzyme amino acid sequences are depicted in SEQ NOs: 17 and 18. SEQ ID NO: 17 is the mature form :whereas SEQ ID NO:18 includes the signal sequence as residues 1-21. In certain embodiments, a proclotting enzyme comprises the amino acid sequence of SEQ ID NO: 8, SEQ ID NO: 9, SEQ ID NO: 17, or SEQ ID NO: 18, or an amino acid sequence having at least 70%, at least 80%, at least 90%, at least 95%, at least 96%, at least 97%, at least 98%, at least 99%, at least 99.5%, or at least 99.8% identity to the amino acid sequence of SEQ D NO: 8, SEQ ID NO: 9, SEQ ID NO: 17, or SEQ ID NO: 18. In certain embodiments, the proclotting enzyme comprises a conservative substitution relative to a wild-type proclotting enzyme sequence or a proclotting enzyme sequence disclosed herein.

In certain embodiments, the proclotting enzyme is a recombinant proclotting enzyme. In certain embodiments, the proclotting enzyme is a native proclotting enzyme, e.g., an isolated native proclotting enzyme.

A contemplated composition may comprise, a for example, from about 0.01 to about 3,000 U/mL, from about 0.01 to about 2,500 U/mL, from about 0.01 to about 2,000 U/mL, from about 0.01 to about 1,500 U/mL, from about 0.01 to about 1,000 U/mL, from about 0.01 to about 500 U/mL, from about 0.01 to about 400 U/mL, from about 0.01 to about 300 U/mL, from about 0.01 to about 200 U/mL, from about 0.01 to about 100 U/mL, from about 0.01 to about 50 U/mL, from about 0.01 to about 20 U/mL, from about 0.01 to about 10 U/mL, from about 0.01 to about 5 U/mL, from about 0.01 to about 2 U/mL, from about 0.01 to about 1 U/mL, from about 0.01 to about 0.5 U/mL, from about 0.01 to about 0.1 U/mL, from about 0.1 to about 3,000 U/mL, from about 0.1 to about 2,500 U/mL, from about 0.1 to about 2,000 U/mL, from about 0.1 to about 1,500 U/mL, from about 0.1 to about 1,000 U/mL, from about 0.1 to about 500 U/mL, from about 0.1 to about 400 U/mL, from about 0.1 to about 300 U/mL, from about 0.1 to about 200 U/mL, from about 0.1 to about 100 U/mL, from about 0.1 to about 50 U/mL, from about 0.1 to about 20 U/mL, from about 0.1 to about 10 U/mL, from about 0.1 to about 5 U/mL, from about 0.1 to about 2 U/mL, from about 0.1 to about 1 U/mL, from about 0.1 to about 0.5 U/mL, from about 0.5 to about 3,000 U/mL, from about 0.5 to about 2,500 U/mL, from about 0.5 to about 2,000 U/mL, from about 0.5 to about 1,500 U/mL, from about 0.5 to about 1,000 U/mL, from about 0.5 to about 500 U/mL, from about 0.5 to about 400 U/mL, from about 0.5 to about 300 U/mL, from about 0.5 to about 200 U/mL, from about 0.5 to about 100 U/mL, from about 0.5 to about 50 U/mL, from about 0.5 to about 20 U/mL, from about 0.5 to about 10 U/mL, from about 0.5 to about 5 U/mL, from about 0.5 to about 2 U/mL, from about 0.5 to about 1 U/mL, from about 1 to about 3,000 U/mL, from about 1 to about 2,500 U/mL, from about 1 to about 2,000 U/mL, from about 1 to about 1,500 U/mL, from about 1 to about 1,000 U/mL, from about 1 to about 500 U/mL, from about 1 to about 400 U/mL, from about 1 to about 300 U/mL, from about 1 to about 200 U/mL, from about 1 to about 100 U/mL, from about 1 to about 50 U/mL, from about 1 to about 20 U/mL from about 1 to about 10 U/mL, from about 1 to about 5 U/mL, from about 1 to about 2 U/mL, from about 2 to about 3,000 U/L, from about 2 to about 2,500 U/mL, from about 2 to about 2,000 U/mL, from about 2 to about 1,500 U/mL, from about 2 to about 1,000 from about 2 to about 500 from about 2 to about 400 U/mL, from about 2 to about 300 U/mL, from about 2 to about 200 U/mL, from about 2 to about 100 U/mL, from about 2 to about 50 U/mL, from about 2 to about 20 U/mL, from about 2 to about 10 U/mL, from about 2 to about 5 U/mL, from about 5 to about 3,000 U/mL, from about 5 to about 2,500 U/mL, from about 5 to about 2,000 U/mL, from about 5 to about 1,500 U/mL from about 5 to about 1,000 U/mL, from about 5 to about 500 U/mL, from about 5 to about 400 U/mL, from about 5 to about 300 U/mL, from about 5 to about 200 U/mL, from about 5 to about 100 U/mL, from about 5 to about 50 U/mL, from about 5 to about 20 U/mL, from about 5 to about 10 U/mL, from about 10 to about 3,000 U/mL, from about 10 to about 2,500 U/mL, from about 10 to about 2,000 U/mL, from about 10 to about 1,500 U/mL, from about 10 to about 1,000 U/mL, from about 10 to about 500 U/mL, from about 10 to about 400 U/mL, from about 10 to about 300 U/mL, from about 10 to about 200 U/mL, from about 10 to about 100 U/mL, from about 10 to about 50 U/mL, from about 10 to about 20 U/mL, from about 20 to about 3,000 U/mL, from about 20 to about 2,500 U/mL, from about 20 to about 2,000 U/mL, from about 20 to about 1,500 U/mL, from about 20 to about 1,000 U/mL, from about 20 to about 500 U/mL, from about 20 to about 400 U/mL, from about 20 to about 300 U/mL, from about 20 to about 200 U/mL, from about 20 to about 100 U/mL, from about 20 to about 50 U/mL, from about 50 to about 3,000 U/mL, from about 50 to about 2,500 U/mL, from about 50 to about 2,000 U/mL, from about 50 to about 1,500 U/mL, from about 50 to about 1,000 U/mL, from about 50 to about 500 U/mL, from about 50 to about 400 U/mL, from about 50 to about 300 U/mL, from about 50 to about 200 U/mL, from about 50 to about 100 U/mL, from about 100 to about 3,000 U/mL, from about 100 to about 2,500 U/mL, from about 100 to about 2,000 U/mL, from about 100 to about 1,500 U/mL, from about 100 to about 1,000 U/mL, from about 100 to about 500 U/mL, from about 100 to about 400 U/mL, from about 100 to about 300 U/mL, from about 100 to about 200 U/mL, from about 200 to about 3,000 from about 200 to about 2,500 U/mL, from about 200 to about 2,000 U/mL, from about 200 to about 1,500 U/mL, from about 200 to about 1,000 U/mL, from about 200 to about 500 U/mL, from about 200 to about 400 U/mL, from about 200 to about 300 U/mL, from about 300 to about 3,000 U/mL, from about 300 to about 2,500 U/mL, from about 300 to about 2,000 U/mL, from about 300 to about 1,500 U/mL, from about 300 to about 1,000 U/mL, from about 300 to about 500 U/mL, from about 300 to about 400 U/mL, from about 400 to about 3,000 U/mL, from about 400 to about 2,500 U/mL, from about 400 to about 2,000 U/mL, from about 400 to about 1,500 U/mL, from about 400 to about 1,000 U/mL from about 400 to about 500 U/mL, from about 500 to about 3,000 U/mL, from about 500 to about 2,500 U/mL, from about 500 to about 2,000 U/mL, from about 500 to about 1,500 U/mL, from about 500 to about 1,000 U/mL, from about 1,000 to about 3,000 U/mL, from about 1,000 to about 2,500 U/mL, from about 1,000 to about 2,000 U/mL, from about 1,000 to about 1,500 U/mL, from about 1,500 to about 3,000 U/mL, from about 1,500 to about 2,500 U/mL, from about 1,500 to about 2,000 U/mL, from about 2,000 to about 3,000 U/mL, from about 2,000 to about 2,500 U/mL, or from about 2,500 to about 3,000 U/mL, of proclotting enzyme (e.g., recombinant proclotting enzyme). Proclotting enzyme enzymatic activity may be assayed by any method known in the art, including, for example, by measuring cleavage of the chromogenic substrate Ac-Ilu-Glu-Gly-Arg-pNA, for example, as described in Example 1 hereinbelow. As used herein one unit (U) of proclotting enzyme activity is defined as the amount of enzyme that catalyzes the conversion of 1 micromole of a substrate (e.g., Ac-Ilu-Glu-Gly-Arg-pNA) per minute.

### E. Factor C

As used herein, the term “factor C” refers to a zymogen, or a functional fragment thereof, that is capable of being activated by endotoxin, and is capable of cleaving (e.g., enzymatically cleaving) factor B to form an activated factor B. The term factor C includes variants having one or more amino acid substitutions, deletions, or insertions relative to a wild-type factor C sequence, and/or fusion proteins or conjugates including a factor C. As used herein, the term “functional fragment” of a factor C refers to fragment of a full-length factor C that retains, for example, at least 10%, at least 20%, at least 30%, at least 40%, at least 50%, at least 60%, at least 70%, at least 80%, at least 90%, at least 95%, at least 96%, at least 97%, at least 98%, at least 99%, or 100% of the enzymatic activity of the corresponding full-length, naturally occurring factor C. Factor C enzymatic activity may be assayed by any method known in the art, including, for example, by measuring cleavage of the chromogenic substrate Z-Val-Pro-Arg-pNA, for example, as described in Example 1 herein. In certain embodiments, the functional fragment comprises at least 100, 200, 300, 400, 500, 600, 700, 800, 900, or 1,000 consecutive amino acids present in a full-length, naturally occurring factor C.

It is contemplated that any horseshoe crab factor C, for example, a Limulus polyphemus, Tachypleus tridentatus, Tachypleus gigas, or Carcinoscorpius rotundicauda factor C, may be used in the practice of the invention.

A DNA sequence encoding an exemplary Limulus polyphemus factor C is depicted in SEQ ID NO: 1. Exemplary Limulus polyphemus factor C amino acid sequences are depicted in SEQ ID NOs: 2 and 3. SEQ ID NO: 2 is the mature form whereas SEQ ID NO:3 includes the signal sequence as residues 1-25. A DNA sequence encoding an exemplary Tachypleus tridentatus factor C is depicted in SEQ ID NO: 10. Exemplary Tachypleus tridentatus factor C amino acid sequences are depicted in SEQ ID NOs: 11 and 12. SEQ ID NO: 11 is the mature form whereas SEQ ID NO: 12 includes the signal sequence as residues 1-21. In certain embodiments, a factor C comprises the amino acid sequence of SEQ ID NO: 2, SEQ ID NO: 3, SEQ ID NO: 11, or SEQ ID NO: 12, or an amino acid sequence having at least 70%, at least 80%, at least 90%, at least 95%, at least 96%, at least 97%, at least 98%, at least 99% identity, at least 99.5%, or at least 99.8% to the amino acid sequence of SEQ ID NO: 2, SEQ ID NO: 3, SEQ II) NO: 1 or SEQ ID NO: 12. In certain embodiments, the factor C comprises a conservative substitution relative to a wild-type factor C sequence or a factor C sequence disclosed herein.

In certain embodiments, the factor C is a recombinant factor C. In certain embodiments, the factor C is a native factor C, e.g., an isolated native factor C.

In certain embodiments, a recombinant horseshoe crab factor C for use as an endotoxin detection reagent, either alone, or in combination with other horseshoe crab proteins (e.g., factor B, proclotting enzyme and/or factor C, whether sourced naturally or produced recombinantly) is expressed in a mammalian cell, for example, a Chinese hamster ovary (CHO) or human embryonic kidney (HEK) cell. In certain embodiments, the recombinant factor C is glycosylated differently (e.g., has one or more different glycosyl groups or a glycosylation pattern) when compared to factor C present in the native horseshoe crab amebocyte lysate, or when compared to factor C produced recombinantly in distinct expression host cells. As a non-limiting example, factor C may be produced recombinantly in a gene-edited cell line, such as GnTI-HEK cells (e.g., HEK cells that do not have N-acetylglucosaminytransferase I (GnTI) activity) that produce factor C proteins lacking (α-2,3)-linked terminal sialic acid. Use of such readily available gene-edited cell lines may be desirable for high-yield expression of homogenously glycosylated recombinant proteins.

### F. Methods of Making Recombinant Proteins

Methods for producing recombinant proteins known in the art. For example, DNA molecules encoding a protein of interest (e.g., factor B, proclotting enzyme and/or factor C, as disclosed herein) can be synthesized chemically or by recombinant DNA methodologies. The resulting DNA molecules encoding the protein of interest can be ligated to other appropriate nucleotide sequences, including, for example, expression control sequences, to produce conventional gene expression constructs (i.e., expression vectors) encoding the desired protein. Production of defined gene constructs is within routine skill in the art.

Nucleic acids encoding desired proteins (e.g., factor B, proclotting enzyme and/or factor C, as disclosed herein) can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma. Transformed host cells can be grown under conditions that permit the host cells to express the genes that encode the protein of interest.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed protein may be secreted. The expressed protein may accumulate in refractile or inclusion bodies, which can be harvested after disruption of the cells by French press or sonication. The refractile bodies then are solubilized, and the protein may be refolded and/or cleaved by methods known in the art.

If the engineered gene is to be expressed in eukaryotic host cells, e.g., CHO or HEK cells, it is first inserted into an expression vector containing a suitable eukaryotic promoter, a secretion signal, a poly A sequence, and a stop codon. Optionally, the vector or gene construct may contain enhancers and introns. The gene construct can be introduced into eukaryotic host cells using conventional techniques.

A polypeptide or protein of interest (e.g., factor B, proclotting enzyme and/or factor C, as disclosed herein) can be produced by growing (culturing) a host cell transfected with an expression vector encoding such a polypeptide or protein, under conditions that permit expression of the polypeptide or protein. Following expression, the polypeptide can be harvested and purified or isolated using techniques known in the art, e.g., affinity tags such as glutathione-S-transferase (GST) or histidine tags.

Provided herein are isolated nucleic acids comprising the sequence of SEQ ID NO: 1, SEQ ID NO: 4, SEQ ID NO: 7, SEQ ID NO: 10, SEQ ID NO: 13, or SEQ ID NO: 16. These sequences can be spliced into a suitable expression vector and operatively linked to a promoter for use in a desired expression host using standard recombinant DNA methodologies. Also provided are expression host cells (e.g., mammalian host cells) comprising such an expression vector, which can then be used to express the protein encoded by one or more of the foregoing nucleic acid sequences. Exemplary methods for making recombinant factor B, proclotting enzyme and factor C are described in Example 1 herein.

In one embodiment, provided herein is a method of producing a horseshoe crab recombinant factor C protein, the method comprising: (a) expressing a nucleic acid sequence encoding the recombinant horseshoe crab factor C in a host cell (e.g., a HEK293 cell or a CHO cell) engineered to remove a glycosyltransferase enzyme (e.g., N-acetylglucosaminyltransferase); and (b) purifying the recombinant factor C expressed in the host cell. Also provided is a recombinant horseshoe crab factor C protein (e.g., a factor C protein that does not contain (α-2,3)-linked terminal sialic acid) produced by such a method. The resulting recombinant horseshoe crab factor C can be used to prepare an endotoxin detection reagent (e.g., a hybrid lysate), by admixing such a recombinant factor C with a composition comprising recombinant horseshoe crab factor B and recombinant horseshoe crab proclotting enzyme.

It is understood that a recombinantly expressed polypeptide or protein may have a different molecular weight and/or be differently glycosylated relative to a corresponding native polypeptide or protein. Similarly, a protein or polypeptide recombinantly expressed in a first host cell type may have a different molecular weight and/or be differently glycosylated relative to a corresponding protein or polypeptide expressed in a second, different host cell type. Glycosylation of recombinant proteins produced in mammalian host cells is described, for example, in Lis et al. (1993) EUR. J. BIOCHEM. 218:1-27, Parodi (2000) ANNU. REV. BIOCHEM. 69:69-93, Viswanathan et al. (2005) BIOCHEM. 44:7526-7534, Tomiya et al. (2004) GLYCOCONJUGATE JOURNAL 21: 343-360, Tomiya et al. (2003) ACC. CHEM. RES. 36:613-620, Gerngros (2004) NAT. BIOTECHNOL. 22:1409-1414, and Demain et al. (2009) BIOTECHNOLOGY ADVANCES 27:297-306.

### G. Methods of Making Hybrid Lysate

Hybrid amebocyte lysates may be produced by combining native and recombinant components disclosed herein.

The invention provides a method for preparing a microbial contaminant (e.g., endotoxin) detection reagent, for increasing the sensitivity of a native horseshoe crab amebocyte lysate for a microbial contaminant (e.g., endotoxin), and/or reducing the amount of a native horseshoe crab amebocyte lysate required to detect a microbial contaminant (e.g., endotoxin). The method comprises adding a recombinant horseshoe crab factor B and/or a recombinant horseshoe crab proclotting enzyme to a native horseshoe crab amebocyte lysate. In certain embodiments, the method comprises diluting the native horseshoe crab amebocyte lysate. For example, a contemplated method comprises diluting a native horseshoe crab amebocyte lysate to produce a diluted horseshoe crab amebocyte lysate; and adding to the diluted native horseshoe crab amebocyte lysate a recombinant horseshoe crab factor B and/or a recombinant horseshoe crab proclotting enzyme. In certain embodiments, the native horseshoe crab amebocyte lysate is diluted by 10%, 20%, 30%, 40%, 50%, 60%, 70%, 80%, 90%, 100%, 150%, 200%, 250%, 300%, 350%, 400%, 450%, 500%, 600%, 700%, 800%, 900%, or 1,000%.

Hybrid amebocyte lysate compositions preferably are sterile. Sterilization can be accomplished by any suitable method, e.g., filtration through sterile filtration membranes. Where the composition is lyophilized, filter sterilization can be conducted prior to or following lyophilization and reconstitution.

Hybrid amebocyte lysate compositions can also be dried onto a solid surface (such as a vial, a cartridge or a 12-well or 96-well plate), such as by lyophilization. Prior to drying, one or more additives are optionally admixed with the hybrid amebocyte lysate. For example, a resolubilizing and/or an anti-flaking agent can be included. The resolubilizing agent is an agent that, either alone or in combination with another resolubilizing agent, facilitates the resolubilization of one or more components of the hybrid amebocyte lysate once the hybrid amebocyte lysate is exposed to a fluid sample. The resolubilizing agent preferably also stabilizes the lysate in its dried form. The resolubilizing agent provided in the mixture facilitates the stability of the reagents and their dissolution during the assay. Resolubilizing agents include, for example, one or more sugars, salts, or combinations thereof Preferred sugar resolubilizing agents include, for example, mannitol, mannose, sorbitol, trehalose, maltose, dextrose, sucrose, and other monosaccharides or disaccharides. The anti-flaking agent included in the mixture further stabilizes the reagents and reduces flaking of the dried lysate. The anti-flaking agent preferably also stabilizes the lysate in its dried form. Preferred anti-flaking agents include, for example, one or more polymers, for example, polyethylene glycol, polyvinyl pyrolidone, polyvinyl alcohol, mannitol, dextran, and proteins, for example, serum albumin. An anti-frothing agent such as polyvinyl alcohol or polypropylene glycol can also be included. Salts and/or buffers, such as sodium chloride, magnesium sulfate, and HEPES buffer can also be included. Other kinds of buffers, such as TRIS-HCl buffer, TES, MOPS, PIPES, BES, MOPSO, DIPSO, MOBS, TAPSO, HEPPSO, POPSO, TEA, EPPS, Tricine, and phosphate can be used, as can other buffers with buffering capacity between pH 7 and pH 8, as this is a preferred range of pH for the reaction. The target pH of the composition, after admixture with a sample, is preferably between 7.3 and 8.0.

The mixture can be dried onto a surface of the conduit in an environment having a temperature of about 4° C. to about 40° C., more preferably, from about 10° C. to about 35° C., more preferably, from about 15° C. to about 30° C. and a relative humidity of about 0% to about 30%, more preferably, from about 2% to about 20%, more preferably, from about 4% to about 10%. Preferably, the temperature is about 25° C. and the relative humidity is about 5%. Drying preferably occurs for about 10 minutes to about S hours, more preferably for about 1 hour in a temperature regulated drying chamber.

In another embodiment, the mixture is dried onto the surface of the conduit by lyophilization or freeze-drying, for example, at temperatures below 0° C., for example, from about −75° C. to about −10° C., more preferably from about −30° C. to about −20° C.

### II. Methods of Detection and Sample Preparation Considerations

Hybrid amebocyte lysates disclosed herein can be used in various assays to detect a microbial contaminant (e.g., endotoxin). The invention provides a method of detecting the presence and/or amount of a microbial contaminant (e.g., endotoxin) in a sample. The method comprises contacting a hybrid amebocyte lysate (e.g., a hybrid amebocyte lysate disclosed herein) with a sample (e.g., a sample suspected of containing endotoxin), allowing the hybrid amebocyte lysate to react with the sample to produce a detectable product (e.g., a gel, increase in turbidity, or a colored or light-emitting product), and detecting the detectable product (e.g., visually or by the use of an optical detector).

Hybrid amebocyte lysates disclosed herein can be used to detect a microbial contaminant (e.g., endotoxin) using, for example, endpoint or kinetic assays. Exemplary endpoint assays include an endpoint chromogenic assay or an endpoint turbidimetric assay. Exemplary kinetic assays include a kinetic turbidimetric assay, a one-step kinetic assay or a multi-step kinetic assay. Each of the assays is discussed in more detail below. Furthermore, it is understood that the assays may be modified to be performed in a particular assay format, for example, in a cartridge or in the well of a plate, for example, a 96 well plate.

### A. Kinetic Assays

Exemplary kinetic assays include multi-step kinetic assays, single-step kinetic assays, and kinetic turbidimetric assays.

### (i) Multi-Step Kinetic Assay

A multi-step kinetic assay (for example, as described in U.S. Pat. No. 7,329,538) is initiated by combining the sample to be tested with a volume of hybrid amebocyte lysate to produce a sample-hybrid amebocyte lysate mixture. The mixture then is incubated for a predetermined period of time. The mixture then is contacted with a substrate, for example, a chromogenic or fluorogenic substrate, to produce a sample-hybrid amebocyte lysate-substrate mixture. Thereafter, the time in which a preselected change in an optical property (for example, a specific change in an absorbance value or a specific change in a transmission value) is measured.

The assay can be calibrated by measuring the time in which a preselected change in an optical property occurs when a certain amount of a microbial contaminant (e.g., endotoxin) is introduced into the assay. By comparing the result generated by a test sample against the results generated by one or more known amounts of the microbial contaminant (e.g., endotoxin), it is possible to detect the presence or amount of the microbial contaminant (e.g., endotoxin) a test sample.

It is understood that a multi-step kinetic assay can be run in a cartridge format. The cartridge preferably is used with an optical detector, for example, a hand-held optical detector as shown and described in U.S. Pat. No. Des. 390,661.

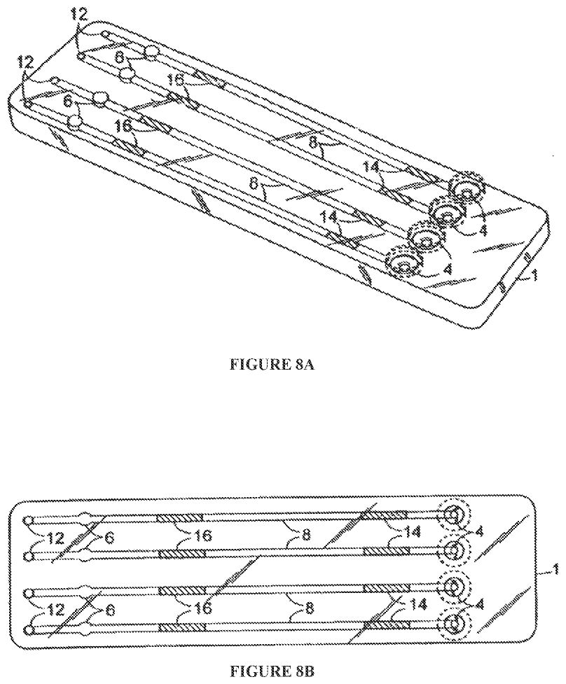

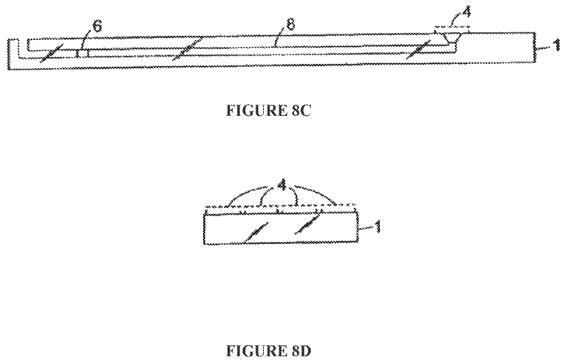

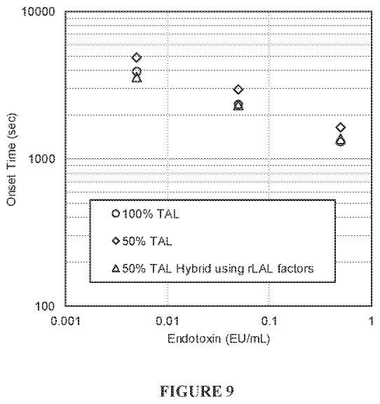

By way of example and as illustrated in FIGS. 8A-8D, cartridge 1 has a substantially planar housing fabricated, for example, from a moldable biocompatible material. The housing may be fabricated from any material, however, transparent and/or translucent glass or polymers are preferred. Preferred polymers include, for example, polystyrene, polycarbonate, acrylic, polyester, optical grade polymers, or any plastic such that the optical cell is substantially transparent. The housing contains at least one fluid inlet port 4, at least one optical cell 6, and at least one conduit 8 having a fluid contacting surface for providing fluid flow communication between the fluid inlet port 4 and optical cell 6. The only requirements for the optical cell 6 are that it defines a void capable of containing a sample to be tested and that a portion of the optical cell 6 is transparent to light. Cartridge 1 may also have at least one pump port 12 in fluid flow communication with fluid inlet port 4 and optical cell 6 for attaching the cartridge 1 to a pump. The pump may then impart a negative pressure via pump port 12 to pull the sample from fluid inlet port 4 to optical cell 6. A hybrid amebocyte lysate is disposed on a first region 14 of the fluid contacting surface of conduit 8, so that when a sample is applied to fluid inlet port 4, the sample traverses region 14 and solubilizes or reconstitutes the hybrid amebocyte lysate into the sample as it moves toward optical cell 6.

A second region 16 of the fluid contacting surface of conduit 8 is spaced apart from and downstream of first region 14. In this configuration, hybrid amebocyte lysate is disposed at first region 14 and a chromogenic or fluorogenic substrate is disposed at second region 16, so that after the sample is contacted with the hybrid amebocyte lysate in region 14, the sample-lysate mixture traverses conduit 8 and contacts the substrate in region 16. The sample-lysate-substrate mixture then traverses conduit 8 to optical cell 6.

The cartridges can be designed and used according to the type and/or number of tests required. For example, a single sample may be tested, for example, in duplicate or triplicate, for example, for research laboratory use or for medical device and biopharmaceutical testing. Alternatively, two or more different samples may be tested individually. The cartridge preferably is a single-use, disposable cartridge that is discarded after one use. The cartridges typically use approximately 20-100 fold less hemocyte lysate per sample than is used in the conventional endpoint chromogenic or kinetic chromogenic assays performed in multi-well plates, and thus provides a less costly and environmentally-friendlier test.

With reference to FIG. 8A, in order to perform a multi-step kinetic assay in an exemplary cartridge 1, a sample is first moved, for example, by pump action, to a first region 14 containing the hybrid amebocyte lysate, where it is mixed and incubated for a predetermined period of time. The sample-hybrid amebocyte lysate mixture then is moved, for example, by pump action, to the second region 16 containing the substrate, for example, a chromogenic or fluorogenic substrate, where it is solubilized. The sample-substrate mixture then is moved to optical cell 6, for a measurement of an optical property. The time intervals required for mixing and incubating steps are preprogrammed for optimal specificity and sensitivity to the microbial contaminant (e.g., endotoxin) concentration range of interest.

Although the multi-step assay may be performed in a cartridge of the type discussed above, it may also be employed in a variety of other formats, for example, within the well of a microtiter plate. In this type of assay, a sample of interest is combined with a hybrid amebocyte lysate and incubated for a predetermined period of time. Then, after the predetermined period of time, a chromogenic or fluorogenic substrate is added to the well. After mixing, the time in which a preselected change in an optical property occurs is measured. The result can then be compared against one or more standard values to measure the presence or amount of a microbial contaminant (e.g., endotoxin) in the sample.

In the well-type format, the samples and reagents are added to each of the wells, preferably using an automated system, such as a robot, and the plate processed by a microplate reader, which can be programmed to sequentially read the absorbance of each well in a repetitive fashion.

### (ii) Single-Step Kinetic Assay

A single-step kinetic assay, for example, a single step-chromogenic assay, is described in U.S. Pat. No. 5,310,657. Briefly, a kinetic chromogenic assay includes the steps of (i) simultaneously solubilizing a hybrid amebocyte lysate with a sample to be analyzed and a substrate, for example, a chromogenic substrate, (ii) incubating the resulting mixture at a temperature of about 0° to about 40° C., preferably about 25° to about 40° C., over a predetermined time range and (iii) measuring a time required for a calorimetric change to reach a pre-selected value or change of the calorimetric readout, using a conventional spectrophotometer.

This type of assay, like the multi-step kinetic assay, can be performed in a cartridge or a well-type format. A cartridge similar to that described above for the multi-step kinetic assay can be modified for use in single-step kinetic assay. With reference to FIG. 8A, chromogenic or fluorogenic substrate is applied, for example, to the surface of conduit 8 at first region 14 together with the hybrid amebocyte lysate. To perform a kinetic assay in cartridge 1 and in reference to FIG. 8A, a sample is moved, for example, by pump action, to a first region 14 of the conduit 8 containing both the hybrid amebocyte lysate and substrate, where they are solubilized, for example, by cycling between forward and reverse pump action. The sample-hybrid amebocyte lysate-substrate mixture then is moved to optical cell 6 for measurement of an optical property, for example, the absorbance or transmittance properties of the sample by an optical detector. The detector may determine how long it takes for each optical property to exhibit, for example, a 5% drop in optical transmittance. Results from multiple assays, for example, two assays, can be averaged.

The assay can be calibrated by measuring the time in which a preselected change in an optical property occurs when a certain amount of a microbial contaminant (e.g., endotoxin) is introduced into the assay. By comparing the result generated by a test sample against one or more results with known amounts of the microbial contaminant (e.g., endotoxin), it is possible to measure the presence or amount of the microbial contaminant (e.g., endotoxin) in the test sample.

This type of assay format may be employed in a variety of other formats, for example, within the well of a microtiter plate. In this type of assay, a sample of interest is mixed with a hybrid amebocyte lysate and a chromogenic or fluorogenic substrate. After mixing, the time in which a preselected change in an optical property occurs is measured. The result can then be compared against standard values to measure the presence or amount of a microbial contaminant (e.g., endotoxin) in the sample of interest.

(iii) Kinetic Turbidimetric Assay

A kinetic turbidimetric assay can include the steps of (i) solubilizing a hybrid amebocyte lysate with a sample to be analyzed, (ii) incubating the resulting mixture at a temperature of about 0° to about 40° C. preferably about 25° to about 40° C., over a predetermined time range, and (iii) measuring a time required for either a turbidity change caused by coagulation to reach a pre-selected value or a ratio in change of the turbidity, using a conventional coagulometer, nepherometer, or spectrophotometer.

This type of assay, like the previous assays, can be performed in a cartridge or a well-type format. A cartridge similar to that described above for the multi-step or single-step kinetic assays can be modified for use in kinetic turbidimetric assays. With reference to FIG. 8A, no chromogenic or fluorogenic substrate needs to be applied to either first region 14 or second region 16.

Referring to FIG. 8A, in order to perform a kinetic turbidimetric assay in a cartridge 1, a sample is, for example, moved to a first region 14 of the conduit 8 containing the hybrid amebocyte lysate, where it is solubilized, for example, by cycling between forward and reverse pump action. The sample-lysate mixture then is moved to optical cell 6 for measurement of an optical property, for example, turbidity, by measuring, for example, the absorbance or transmittance properties of the sample-lysate mixture using an optical detector. The detector may determine how long it takes for each optical property to exhibit, for example, a 5% drop in optical transmittance. Results from multiple assays, for example, two assays can be averaged.

The assay can be calibrated by measuring the time in which a preselected change in an optical property, for example, turbidity, occurs when a certain amount of a microbial contaminant (e.g., endotoxin) is introduced into the assay. By comparing the result generated by a test sample against one or more results with known amounts of the microbial contaminant (e.g., endotoxin), it is possible to measure the presence or amount of the microbial contaminant (e.g., endotoxin) in the test sample.

This type of assay format may be employed in a variety of other formats, for example, within the well of a microtiter plate. In this type of assay, a sample of interest is mixed with a hybrid amebocyte lysate. After mixing, the time in which a preselected change in an optical property, for example, turbidity, occurs is measured. The result can then be compared against standard values to measure the presence or amount of a microbial contaminant (e.g., endotoxin) in the sample of interest.

### B. Endpoint Assays

Exemplary endpoint assays include endpoint chromogenic or fluorogenic and endpoint turbidimetric assays.

### (i) Endpoint Chromogenic or Fluorogenic Assay

Endpoint chromogenic or fluorogenic assays can include the steps of (i) solubilizing a hybrid amebocyte lysate with a sample to be analyzed, (ii) incubating the resulting mixture at a temperature of about 0° to about 40° C., preferably about 25° to about 40° C., for a predetermined time, (iii) contacting substrate, for example, a chromogenic or fluorogenic substrate, with the incubated sample-hybrid amebocyte lysate mixture, (iv) optionally adding a reaction inhibitor, for example, acetic acid, and (v) measuring, for example by calorimetric change, a substance produced from the substrate by enzymatic activity.

This type of assay can be performed in a cartridge or in a well-type format. When an endpoint chromogenic or fluorogenic assay is performed in a cartridge 1 (see. FIG. 8A), a sample is moved, for example, to a first region 14 of the conduit 8 containing the hybrid amebocyte lysate, where it is solubilized, for example, by cycling between forward and reverse pump action. Following a predetermined incubation period, the sample-hybrid amebocyte lysate mixture then is moved, for example, by pump action to a second region 16 of the conduit 8 containing the chromogenic or fluorogenic substrate, where it is solubilized, for example, by cycling between forward and reverse pump action. The sample-hybrid amebocyte lysate-substrate mixture optionally then is moved to a third region containing a reaction inhibitor. Afterwards, the sample-hybrid amebocyte lysate-substrate mixture is moved to optical cell 6 for measurement of an optical property, for example, the absorbance or transmittance properties of the sample by an optical detector. It is contemplated, however, that when performing an end-point chromogenic or fluorogenic assay in a cartridge it is not necessary to stop the reaction using a reaction inhibitor. Under this type of assay, the final optical readings (endpoint readings are recorded at a predetermined time.

The assay can be calibrated by measuring an optical property, for example, absorbance or transmittance, when a certain amount of a microbial contaminant (e.g., endotoxin) is introduced into the assay. By comparing the result generated by a test sample against one or more results with known amounts of the microbial contaminant (e.g. endotoxin), it is possible to measure the presence or amount of the microbial contaminant (e.g., endotoxin) in the test sample.

As discussed, this type of assay format may be employed in a variety of other formats, for example, within the well of a microtiter plate. In this type of assay, a sample of interest is mixed with a hybrid amebocyte lysate and incubated for a preselected period of time. Then, a chromogenic or fluorogenic substrate is added to the mixture and the sample incubated for another period of time. Then a reaction inhibitor, for example, acetic acid, is added to the sample, and an optical property of the sample, for example, absorbance or transmittance, is measured. The result can then be compared against standard values to measure the presence or amount of a microbial contaminant (e.g., endotoxin) in the sample of interest.

### (ii) Endpoint Turbidimetric Assay

End point turbidimetric assays can include the steps of (i) solubilizing a hybrid amebocyte lysate with a sample to be analyzed, (ii) incubating the resulting mixture at a temperature of about 0° to about 40° C., preferably about 25° to about 40° C., for a predetermined time, (iii) optionally adding a reaction inhibitor, for example, acetic acid, and (iv) measuring the increase in turbidity as a result of coagulation, if any, using a conventional coagulometer, nepherometer, or spectrophotometer.

Endpoint turbidimetric assays can be performed in a cartridge-type format. With reference to FIG. 8A, a sample is applied to cartridge 1 and is moved, for example, to a first region 14 of the conduit 8 containing the hemocyte lysate, where it is solubilized, for example, by cycling between forward and reverse pump action. The sample-lysate mixture then is moved to optical cell 6 for measurement of an optical property, for example, turbidity, using an optical detector. Results from multiple assays, for example, two assays can be averaged.

The assay can be calibrated, for example, by measuring the turbidity at a preselected time when a certain amount of a microbial contaminant (e.g., endotoxin) is introduced into the assay. By comparing the result generated by a test sample against one or more results with known amounts of the microbial contaminant (e.g., endotoxin), it is possible to measure the presence or amount of the microbial contaminant (e.g., endotoxin) in the test sample.

This type of assay format may also be run in other formats, for example, within the well of a microtiter plate. In this type of assay, a sample of interest is mixed with a hybrid amebocyte lysate and incubated for a preselected period of time. The reaction can then be stopped by the addition of an inhibitor. An optical property, for example, turbidity, of the sample then is measured at a preselected time point. The result can then be compared against standard values to measure the presence or amount of the microbial contaminant (e.g., endotoxin) in the sample of interest.

### C. Specimen Collection and Preparation Considerations

In general, materials used to harvest, store, or otherwise contact a sample to be tested, as well as test reagents, should be free of microbial contamination, for example, should be pyrogen-free. Materials may be rendered pyrogen-free by, for example, heating at 250° C. for 30 minutes. Appropriate precautions should be taken to protect depyrogenated materials from subsequent environmental contamination.

The hybrid amebocyte lysate may be used to measure the presence or amount of a microbial contaminant (e.g., endotoxin) in a sample of interest, for example, in a fluid, for example, a fluid to be administered locally or systemically, for example, parenterally to a mammal, or a body fluid to be tested for infection, including, for example, blood, lymph, urine, serum, plasma, ascites fluid, lung aspirants, and the like. In addition, the assays may be used to detect a microbial contaminant (e.g., endotoxin) present on a surface. For example, the surface of interest is swabbed and the swab then is introduced into or dissolved in liquid. The liquid can then be assayed as described herein.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred from the context.

It should be understood that the order of steps or order for performing certain actions is immaterial so long as the present invention remain operable. Moreover, two or more steps or actions may be conducted simultaneously.

At various places in the present specification, variable or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified.

Unless defined otherwise, all technical and scientific terms used herein have the same meaning as commonly understood by one of ordinary skill in the art to which this invention belongs. The abbreviations used herein have their conventional meaning within the chemical and biological arts.

### EXAMPLES

The following Examples are merely illustrative and are not intended to limit the scope or content of the invention in any way.

### Example 1

This Example describes the preparation of recombinant Limulus polyphemus factor C, factor B, and pro-clotting enzyme.

DNA sequences encoding Limulus polyphemus factor C, factor B and pro-clotting enzyme (with codon usage optimized for expression in mammalian cells) were cloned into expression plasmid BD609 (ATUM). The DNA sequence encoding Limulus polyphemus factor C is depicted in SEQ ID NO: 1 (and the corresponding amino acid sequence is depicted in SEQ ID NO: 3). The DNA sequence encoding Limulus polyphemus factor B is depicted in SEQ ID NO: 4 (and the corresponding amino acid sequence is depicted in SEQ ID NO: 6). The DNA sequence encoding Limulus polyphemus pro-clotting enzyme is depicted in SEQ ID NO: 7 (and the corresponding amino acid sequence is depicted in SEQ ID NO: 9). Expression plasmids were transfected into HEK-293 cells using the FreeStyle 293 Expression System (Thermo Fisher) to generate a stable clonal cell lines.

For expression and purification, HEK-293 cells were thawed and added to FreeStyle 293 Expression Media in a flask. Cells were grown at 37° C., 5-7% CO₂ at 120 rpm and passaged every 24-72 hours. When cells reached the desired volume and density, they were used to seed a total of 20 L of culture in a WAVE Bioreactor. After 72 hours, the supernatant was harvested by centrifugation at 4,000×g for 15 minutes followed by sterile filtration. The supernatant was concentrated to <2 L and buffer exchanged by Tangential Flow Filtration (TFF, GE Life Sciences). The TFF system was equilibrated with 20 mM Tris-HCl buffer pH 8.0 containing 20 mM NaCl.

To mitigate endotoxin exposure, all materials used were single use. Water for injection was used for all buffers, and all buffers were made on the day of use.

The amount of each recombinant protein was measured in terms of activity (U/mL) assayed under the following conditions. Recombinant factor C (rFC) was activated by addition of endotoxin (1 μg/mL lipopolysaccharide from E. coli O55:B5 (List Labs)) and incubation at 37° C. for 30 minutes. Then, the cleavage of the synthetic substrate Z-Val-Pro-Arg-pNA was measured by monitoring absorbance at 405 nm at 37° C. Recombinant factor B (rFB) was activated by addition of activated rFC and incubation at 37° C. for 1 hour. Then, the cleavage of the synthetic substrate H-D-Leu-Thr-Arg-pNA was measured by monitoring absorbance at 405 nm at 37° C. Recombinant pro-clotting enzyme (rPCE) was activated by addition of activated rFB and incubation at 37° C. for 1 hour. Then, the cleavage of the synthetic substrate Ac-Ilu-Glu-Gly-Arg-pNA was measured by monitoring absorbance at 405 nm at 37° C. One unit of activity was defined as the amount of enzyme that catalyzed the conversion of 1 micromole of substrate per minute.

### Example 2

This example describes the addition of recombinant Limulus polyphemus factor C, factor B, and/or pro-clotting enzyme to crude Limulus amebocyte lysate (LAL), and the resulting endotoxin detection activity.

Crude LAL was prepared generally as described in Levin et al. (1968) THROMB. DIATH. HAEMORRH. 19:186. Briefly, hemolymph was harvesting from horseshoe crab. The resulting hemolymph was centrifuged to produce an amebocyte pellet. The amebocytes were then re-harvested, re-rinsed, and re-centrifuged. After second rinsing and harvesting steps, the resulting amebocytes were lysed by osmotic shock, and the resulting crude amebocyte lysate was stored at 2-8° C. until further use.

Recombinant Limulus polyphemus factor C (rFC), factor B (rFB), and pro-clotting enzyme (rPCE) were prepared as described in Example 1. A typical LAL endotoxin detection reagent includes 20% crude LAL, appropriate/compatible divalent cations, detergents, excipients for lyophilization, and substrate (Ac-Ilu-Glu-Gly-Arg-pNA). LAL endotoxin detection reagents were generated including reduced amounts of crude LAL (e.g., 10% or 5%), as well as additional recombinant proteins, as shown in TABLE 1.

TABLE 1

| Reagent | Final concentration | | | |
|---|---|---|---|---|
| No. | Crude LAL (%) | rFC (U/mL) | rFB (U/mL) | rPCE (U/mL) |
| 0 | 10 | None | None | None |
| 1 | 10 | 21.4 | None | None |
| 2 | 10 | None | 0.2 | None |
| 3 | 10 | None | 0.11 | 114 |
| 4 | 5 | None | 0.11 | 114 |
| 5 | 5 | 10.7 | 0.11 | 114 |
| 6 | 5 | None | 0.12 | 190 |

Endotoxin was detected using a kinetic chromogenic assay method (KCA). Briefly, 0.1 mL of LAL reagent was mixed with 0.1 U/mL of a sample containing RSE (US Reference Standard Endotoxin). Reaction of endotoxin with the LAL reagent leads to cleavage of the Ac-Ilu-Glu-Gly-Arg-pNA substrate in the LAL reagent and a resulting chromogenic signal that can be measured by absorbance at 405 nm. The activity of the LAL reagent was measured by onset time (the amount of time to reach to reach a particular absorbance (OD 0.05) at 405 nm. Onset time was measured with 0.02-20 EU/mL RSE, and logarithmic plotting of the onset times at each concentration was used to generate a standard curve.

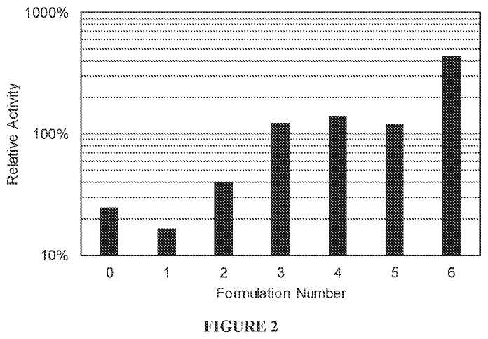

The onset time at 0.2 EU/mL of RSE was compared for different reagents. Results are shown in FIG. 2. Activity in FIG. 2 is relative to an LAL reagent containing undiluted (20%) crude LAL, i.e., 100% activity in FIG. 2 corresponds to the activity (onset time for 0.2 EU/mL of RSE, calculated as described above) of an LAL reagent containing undiluted (20%) crude LAL.

Reagent No. 0 (including diluted (10%) crude LAL, with no additional recombinant proteins) showed 25% activity relative to a reagent including undiluted (20%) crude LAL. Addition of rFc alone (Reagent No. 1) did not increase relative activity. Addition of rFB alone (Reagent No. 2) did increase relative activity. However, the greatest increase in relative activity was observed following the addition of both rFB and rPCE. For certain reagents (Reagent Nos. 4-6) the relative activity was greater than 100%. In other words, even though these reagents contained diluted crude LAL, they showed greater endotoxin detection activity than a corresponding reagent with undiluted crude LAL.

Together, these results show that the addition of rFB and/or rPCE to a native amebocyte lysate (e.g., native LAL) can reduce the amount of native amebocyte lysate required to detect endotoxin (e.g., in a KCA assay) while maintaining, or in certain instances even increasing, sensitivity of the amebocyte lysate.

### Example 3

This example describes the addition of varying amounts of recombinant Limulus polyphemus factor B to crude Limulus amebocyte lysate (LAL), and the resulting impact on endotoxin detection activity.

Recombinant Limulus polyphemus factor B (rFB) and pro-clotting enzyme (rPCE) were prepared as described in Example 1, crude LAL was prepared as described in Example 2, and LAL reagent activity was assayed generally as described in Example 2.

LAL reagents were generated containing diluted crude LAL, rPCE, and varying amounts of rFB, as shown in TABLE 2. The rFB amounts used were 0, 0.2, and 0.4 U/mL.

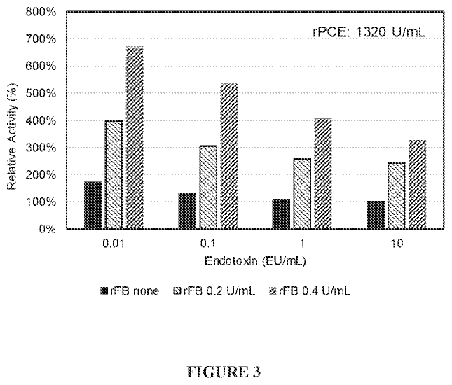

Results are shown FIG. 3 and TABLE 3. FIG. 3 depicts activity for the indicated reagents relative to the activity of Reagent No. 1 (an LAL reagent containing undiluted (20%) crude LAL and no recombinant proteins) at the same endotoxin concentration, i.e., 100% activity in FIG. 3 for a given endotoxin concentration corresponds to the activity of Reagent No. 1 at the same endotoxin concentration. TABLE 3 depicts onset times at the indicated endotoxin concentration for each reagent.

As shown in FIG. 3 and TABLE 3, addition of rPCE alone increased activity, as Reagent No. 2 (with diluted crude LAL and rPCE) had comparable activity to Reagent No. 1 (with undiluted crude LAL and no rPCE). However, the greatest increase in activity was observed following the addition of both rFB and rPCE, as seen in Reagent Nos. 3 and 4, which had substantially increased activity relative to Reagent No. 1. The increase in activity was generally in proportion to the amount of rFB that was added.

TABLE 2

| Reagent | | | | |
|---|---|---|---|---|
| No. | 1 | 2 | 3 | 4 |
| LAL | 100% (20%) | 50% (10%) | 50% (10%) | 50% (10%) |
| rPCE (U/mL) | None | 1320 | 1320 | 1320 |
| rFB (U/mL) | None | None | 0.2 | 0.4 |

TABLE 3

| | |
|---|---|
| Endotoxin | Onset Time (Sec) |

| (EU/mL) | 1 | 2 | 3 | 4 |
|---|---|---|---|---|
| NC | 5999 | 5678 | 5220 | 5820 |
| 0.01 | 4650 | 4035 | 3233 | 2820 |
| 0.1 | 2543 | 2370 | 1905 | 1650 |
| 1 | 1403 | 1373 | 1103 | 985 |
| 10 | 785 | 784 | 630 | 584 |

Together, these results show that the addition of rFB and/or rPCE to a native amebocyte lysate (e.g., native LAL) can reduce the amount of native amebocyte lysate required to detect endotoxin (e.g., in a KCA assay) while maintaining, or in certain instances even increasing, sensitivity of the amebocyte lysate.

### Example 4

This example describes the preparation of lyophilized. LAL reagents containing diluted crude LAL and recombinant Limulus polyphemus factor B (rFB) and pro-clotting enzyme (rPCE).

rFB and rPCE were prepared as described in Example 1, and crude LAL was prepared as described in Example 2. LAL reagents were generated containing diluted crude LAL, rPCE, and rFB as shown in TABLE 4, and were lyophilized.

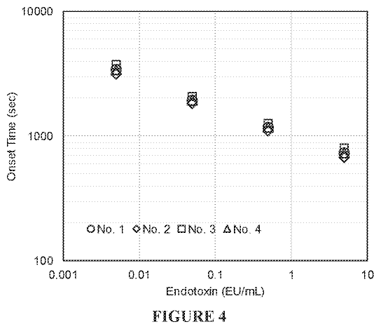

LAL reagent activity was assayed generally as described in Example 2. Results are shown in FIG. 4 and TABLE 5. FIG. 4 shows the onset time at each indicated endotoxin concentration for the reagents, and TABLE 5 shows information on regression curves that result from fitting the data shown in FIG. 4. All formulations showed a dose-dependent response to endotoxin.

TABLE 4

| Reagent | Final concentration | | |
|---|---|---|---|
| No. | Crude LAL (%) | rFB (U/mL) | rPCE (U/mL) |
| 1 | 10 | 0.2 | 50 |
| 2 | 10 | 0.2 | 100 |
| 3 | 10 | 0.1 | 50 |
| 4 | 10 | 0.1 | 100 |

TABLE 5

| Item | No. 1 | No. 2 | No. 3 | No. 4 |
|---|---|---|---|---|
| Slope | −0.222 | −0.223 | −0.222 | −0.221 |
| Y-intercept | 2249 | 2101 | 2440 | 2257 |
| r | −0.999 | −1.00 | −0.998 | −0.999 |

These results indicate that hybrid amebocyte lysates (including, e.g., diluted crude LAI rFB, and/or rPCE) maintain activity following lyophilization.

### Example 5

This example describes the preparation of LAL reagents containing diluted crude LAL, recombinant Limulus polyphemus factor B (rFB), and recombinant Limulus polyphemus pro-clotting enzyme (rPCE) and their use in kinetic turbidimetric (KTA) or gel-clot assays.

rFB and rPCE were prepared as described in Example 1, and crude LAL was prepared as described in Example 2. LAL reagents were generated containing 0.5 U/mL rFB, 100 U/mL rPCE, and 19% crude LAL (which is about half amount of crude lysate in a typical KTA ILL reagent). KTA and gel-clot assays were performed according to the Bacterial Endotoxins Test as described in the United States Pharmacopeia (USP).

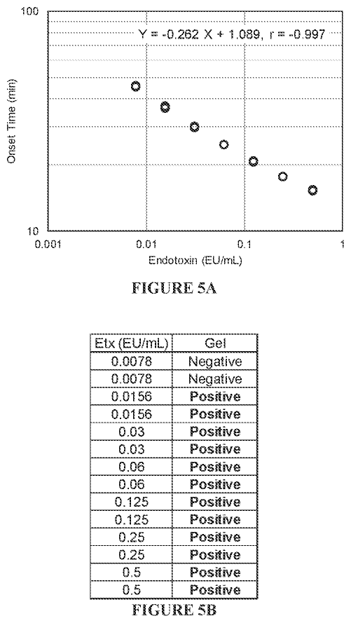

Results for the KTA assay are shown in FIG. 5A and for the gel-clot assay are shown in FIG. 5B. The reagent exhibited a dose dependent response to RSE in the KTA assay, and an endpoint of 0.015 EU/mL in the gel-clot assay.

Together, these results show that the addition of recombinant proteins (e.g., rFB and/or rPCE) to a native amebocyte lysate (e.g., native LAL) can reduce the amount of native amebocyte lysate required to detect endotoxin (e.g., in a KTA or gel-clot assay) while maintaining sensitivity of the amebocyte lysate.

### Example 6

This example describes the preparation of LAL reagents containing further diluted crude LAL, recombinant Limulus polyphemus factor B (rFB), and recombinant Limulus polyphemus pro-clotting enzyme (rPCE) and their use in kinetic turbidimetric (KTA) or gel-clot assays.

rFB and rPCE were prepared as described in Example 1, and crude LAL was prepared as described in Example 2. LAL reagents were generated containing 0.5 U/mL rFB, 100 U/mL rPCE, and 12% crude LAL (which is about 30% the amount of crude lysate in a typical KTA LAL reagent). LAL reagent activity was assayed generally as described in Example 5.

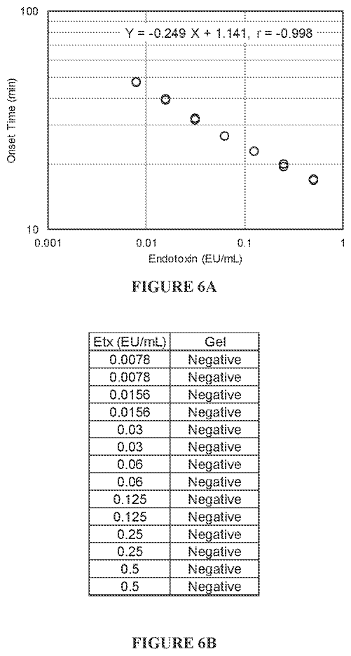

Results for the KTA assay are shown in FIG. 6A and for the gel-clot assay are shown in FIG. 6B. The LAL reagent exhibited a dose dependent response to RSE in the KTA assay, but did not achieve an endpoint in this gel-clot assay.

Together, these results show that the addition of recombinant proteins (e.g., rFB and/or rPCE) to a native amebocyte lysate (e.g., native LAL) can reduce the amount of native amebocyte lysate required to detect endotoxin (e.g., in a KTA) while maintaining sensitivity of the amebocyte lysate.

### Example 7

Natural environmental endotoxins (NEE), including endotoxins in water samples, are different from purified endotoxin, such as RSE prepared from E. coli, and often show low reactivity to recombinant reagents including recombinant Factor C reagents and recombinant LAL (Dubczak et al. (2021) EUR. J. PHRM. SCI. 159:105716). This example describes the preparation of LAL, reagents containing diluted crude LAL, recombinant Limulus polyphemus factor B (rFB), and recombinant Limulus polyphemus pro-clotting enzyme (rPCE) and their use in the detection of NEEs.

The following reagents were tested in this Example: (i) a fully recombinant LAL, including 20 U/mL, rFC, 0.3 U/mL, rFB, and 350 U/mL rPCE (“rLAL”), (ii) a hybrid LAL including diluted (10%) crude LAL, 0.1 U/mL, rFB, and 100 U/mL, rPCE (“hybrid”), (iii) a first commercially available recombinant factor C (PyroGene, Lonza, “rFC #1”), (iv) a second commercially available recombinant factor C (EndoZyme, BioMerieux, “rFC #2”), (v) a first commercially available native LAL reagent (Endochrome-K, Charles River), and (vi) a second commercially available native LAL reagent (Kinetic QCL, Lonza). For rLAL and hybrid, rFc, rFB and rPCE were prepared as described in Example 1, crude LAL was prepared as described in Example 2, and activity was assayed generally as described in Example 2. All commercially available reagents were used according to manufacturer's instructions.

The commercially available native LAI, reagents (Endochrome-K and Kinetic QCL) were used as a positive control. NEE in 114 water samples was detected using both Endochrome-K and Kinetic QCL, and the average of these two values was set to 100%. rLAL, hybrid, rFC #1, and rFC #2 were also used detect NEE in the same 114 water samples, and the results were compared to those obtained using Endochrome-K and Kinetic QCL. According to the criteria in the Bacterial Endotoxins Test (BET) in pharmacopoeias, each reagent was considered to agree with the Endochrome-K and Kinetic QCL average if it differed by no more than a factor of 2 (i.e., ranged from 50% to 200%). The results are shown in TABLE 6.

TABLE 6

| | rFC # 1 | | rFC #2 | | rLAL | | Hybrid | |
|---|---|---|---|---|---|---|---|---|
| Criteria | Subtotal | Ratio | Subtotal | Ratio | Subtotal | Ratio | Subtotal | Ratio |
| <50% | 93 | 81.6% | 111 | 97.4% | 44 | 38.6% | 1 | 0.9% |
| 50%-200% | 21 | 18.4% | 3 | 2.6% | 70 | 61.4% | 110 | 96.5% |
| >200% | 0 | 0.0% | 0 | 0.0% | 0 | 0.0% | 3 | 2.6% |
| Total | 114 | 100% | 114 | 100% | 114 | 100% | 114 | 100% |

As shown in TABLE 6, the hybrid lysate showed 96.5% agreement with the native LAL, reagents (Endochrome-K and Kinetic QCL) while rLAL, rFC #1, and rFC #2 showed high rates of failure. Notably, rFC #1 and rFC #2 reagents failed on the lower side more than 80%, suggesting a high rate of potential false negatives when using these reagents.

Together, these results indicate that hybrid amebocyte lysate reagents (including, e.g., diluted crude LAL, rFB, and/or rPCE) are able to detect NEE at levels comparable to native LAL reagents, and are superior at detecting NEE relative to fully recombinant reagents.

### Example 8

This example describes the addition of recombinant Tachypleus tridentatus factor B, and/or pro-clotting enzyme to crude Tachypleus amebocyte lysate (TAL), and the resulting endotoxin detection activity.

Recombinant Tachypleus tridentatus factor B (rFB) and proclotting enzyme (rPCE) were prepared using the same cloning, expression, and purification methods described in Example 1. The DNA sequence encoding Tachypleus tridenatus factor B is depicted in SEQ ID NO: 13 (and the corresponding amino acid sequence is depicted in SEQ ID NO: 15). The DNA sequence encoding Tachypleus tridentatits pro-clotting enzyme is depicted in SEQ ID NO: 16 (and the corresponding amino acid sequence is depicted in SEQ ID NO: 18).

Commercially available TAL reagent (Zhanjiang A & C Biological Ltd., China) was reconstituted with LAL reagent water and diluted two-fold in a buffer containing 80 mM HEPES buffer (pH 7.6), 1.6% NaCl, 20 mM MgSO₄, and 1 mM Ac-Ilu-Glu-Gly-Arg-pNA. This reagent is referred to as “100% TAL”. A hybrid reagent, referred to as “50% TAL-Hybrid”, was prepared by diluting 100% TAL two-fold and adding 0.1 U/mL, rFB and 100 U/mL rPCE. A control reagent, referred to as “50% TAL” was also prepared by diluting 100% TAL two-fold without the addition of any recombinant proteins.

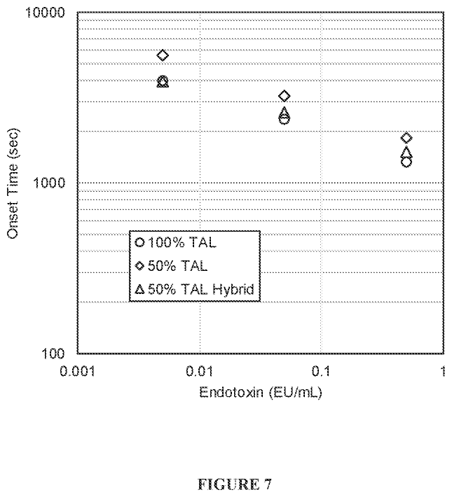

100% TAL, 50% TAL, and 50% TAL-Hybrid were tested in a KCA assay (generally as described in Example 2). Results are shown in FIG. 7. As depicted, the sensitivity of 50% TAL was lower than 100% TAL (with 50% TAL having 25% of the sensitivity of 100% TAL). However, 50% TAL-Hybrid showed similar sensitivity as 100% TAL (with 50% TAL-Hybrid having 79% of the sensitivity of 100% TAL).

100% TAL, 50% TAL, rLAL (as described in Example 7) and 50% TAL-Hybrid were also used to detect NEE in one of the water samples from Example 7. Results are shown in TABLE 7. Endotoxin values measured using 100% TAL were set to 100%. As depicted, 50% TAL-Hybrid had comparable activity to 100% TAL, and greater activity than 50% TAL or rLAL.

TABLE 7

| Reagent | Relative Activity |
|---|---|
| 100% TAL | 100% |
| 50% TAL | 57% |
| rLAL | 9% |
| 50% TAL Hybrid | 88% |

Together, these results show that the addition of rFB and/or rPCE to a native amebocyte lysate (e.g., native TAL) can reduce the amount of native amebocyte lysate required to detect endotoxin (e.g., in a KCA assay) while maintaining, or in certain instances even increasing, sensitivity of the amebocyte lysate.

### Example 9

This example describes the addition of recombinant Limulus polyphemus factor B, and/or pro-clotting enzyme to crude Tachypleus amebocyte lysate (TAL), and the resulting endotoxin detection activity.

Recombinant Limulus polyphemus factor B (rFB) and proclotting enzyme (rPCE) were prepared as described in Example 1. Commercially available TAL reagent (Zhanjiang A & C Biological Ltd., China) was reconstituted with LAL reagent water and diluted two-fold in a buffer containing 80 mM HEPES buffer (pH 7.6), 1.6% NaCl, 20 mM MgSO₄, and 1 mM Ac-Ilu-Glu-Gly-Arg-pNA. This reagent is referred to as “100% TAL”. A hybrid reagent, referred to as “50% TAL-rLP Hybrid”, was prepared by diluting 100% TAL two-fold and adding 0.1 polyphemus rFB and 100 U/mL, Limulus polyphemus rPCE. A control reagent, referred to as “50% TAL” was also prepared by diluting 100% TAL two-fold without the addition of any recombinant proteins.

100% TAL, 50% TAL, and 50% TAL-rLP Hybrid were tested in a KCA assay (generally as described in Example 2). Results are shown in FIG. 9. As depicted, the sensitivity of 50% TAL was lower than 100% TAL (with 50% TAL having 38% of the sensitivity of 100% TAL). However, 50% TAL-rLP Hybrid showed greater sensitivity than 100% TAL (with 50% TAL-rLP Hybrid having 113% of the sensitivity of 100% TAL).

100% TAL, 50% TAL, rLAL (as described in Example 7) and 50% TAL-rLP were also used to detect NEE in one of the water samples from Example 7. Results are shown in TABLE 8. Endotoxin values measured using 100% TAL were set to 100%. As depicted, 50% TAL-rLP had improved activity relative to 50% TAL or rLAL.

TABLE 8

| Reagent | Relative Activity |
|---|---|
| 100% TAL | 100% |
| 50% TAL | 68% |
| rLAL | 9% |
| 50% TAL-rLP Hybrid | 77% |

Together, these results show that the addition of rFB and/or rPCE (e.g., Limulus rFB and/or rPCE) to a native amebocyte lysate (e.g., native TAL) can reduce the amount of native amebocyte lysate required to detect endotoxin (e.g., in a KCA assay) while maintaining, or in certain instances even increasing, sensitivity of the amebocyte lysate.

### Example 10

This example describes the addition of recombinant Tachypleus tridentatus factor B, and/or pro-clotting enzyme to crude Limulus amebocyte lysate (LAL), and the resulting endotoxin detection activity.

Recombinant Tachypleus tridentatus factor B (rFB) and proclotting enzyme (rPCE) were prepared as described in Example 8. Crude LAL was prepared as described in Example 2. This reagent is referred to as “100% LAL”. A hybrid reagent, referred to as “50% LAL-rT Hybrid”, was prepared by diluting 100% LAL two-fold and adding 0.1 U/mL Tachypleus tridentatus rFB and 100 U/mL Tachypleus tridentatus rPCE. A control reagent, referred to as “50% LAL” was also prepared by diluting 100% LAL two-fold without the addition of any recombinant proteins.

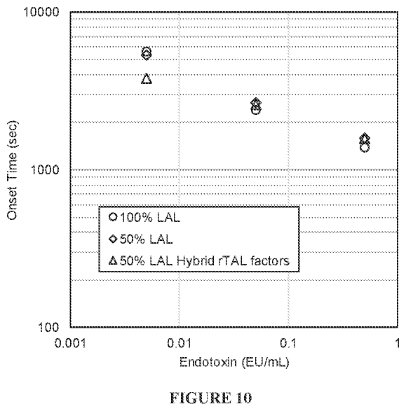

100% LAL, 50% LAL, and 50% LAL-rT Hybrid were tested in a KCA assay (generally as described in Example 2). Results are shown in FIG. 10. As depicted, the sensitivity of 50% LAL was lower than 100% LAL (with 50% LAL having 77% of the sensitivity of 100% LAL). However, 50% LAL-rT Hybrid showed greater sensitivity than 100% LAL (with 50% LAL-rT Hybrid having 129% of the sensitivity of 100% LAL).

100% LAL, 50% LAL, rLAL (as described in Example 7) and 50% LAL-rT Hybrid were also used to detect NEE in one of the water samples from Example 7. Results are shown in TABLE 9. Endotoxin values measured using 100% LAL were set to 100%. As depicted, 50% LAL-rT Hybrid had comparable activity to 100% LAL, and greater activity than 50% LAL or rLAL.

TABLE 9

| Reagent | Relative Activity |
|---|---|
| 100% LAL | 100% |
| 50% LAL | 66% |
| rLAL | 9% |
| 50% LAL-rTAL Hybrid | 91% |

Together, these results show that the addition of rFB and/or rPCE (e.g., Tachypleus tridentatus rFB and/or rPCE) to a native amebocyte lysate (e.g., native LAL) can reduce the amount of native amebocyte lysate required to detect endotoxin e.g., in a KCA assay) while maintaining, or in certain instances even increasing, sensitivity of the amebocyte lysate.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

SEQUENCE LISTING

SEQ ID NO: 1
ATGGTGCTTGCCTCCTTTCTCGTGTCCGGTCTTGTGCTCGGCCTCCTCGCCCAAAAGATGCGAC
CCGTGCAGTCCAGAGGAGTGGACCTGGGCCTGTGCGACGATACCCGGTTCGAGTGCAAATGCGG
CGACCCGGGTTACGTGTTCAATGTGCCTGCGAAGCAGTGTACCTACTTTTACCGCTGGCGCCCT
TACTGCAAGCCATGCGACAAGCTTGAGGCTAAAGATGTGTGCCCCAAGTACAAGAGGTGCCAAG
AATGCCGCGCCGGCCTGGATTCATGCGTGAGCTGCCCCCCAAACAAATACGGCACTTGGTGCTC
CGGGGAGTGCCAGTGCAAGAACGGCGGAATCTGCGACCAGAGGACCGGGGCCTGCACTTGCCGG
GACCGCTACGAGGGGGTGCATTGCGAGATCCTGCAGGGTTGCCCGCTGCTGCAGTCCGACCCCC
AAGTGCAGGAGGTCAAGAACCCCCCCAACGACCCGCAGACCATCGACTACTCCTGTTCACCTGG
GTTCAAGCTGAAGGGCGTGGCACGGATTACGTGCCTCCCCAACGGACAGTGGTCATCCTTCCCC
CCTAAGTGCATTCGGGAGTGCAGCATGGTGTCGAGCTTGGAACACGGAAAGGTCAACTCCCCGA
GCGCCGACCTGATTGAGGGGGCGACCCTTCGGTTCTCGTGCGACTCCCCCTATTACCTGATCGG
TCAGGAAACCCTGACGTGCCAGGGCAACGGCCAATGGTCGGGGCAGATCCCGCAGTGTCAGAAG
CTCGTGTTTTGTCCCGATTTGGACCCAGTGTCCCATGCCGAACACCAGGTCAAGATCGGACTGG
AACAGAAATACGGACAGTTCCCCCAAGGCACTGAAGTCACTTACACTTGCACCGGAAACTACTT
CCTGATGGGCCTGGACACCCTCAAGTGCAATCCCGACGGATCCTGGTCGGGCACTCAGCCGTCC
TGCGTGAAAGTGGCAGACAGAGAAGTGAACTGTGATAGCAAAGCTGTGGACTTCCTGGACGACG
TGGGCGAACCGGTCCGCATCCACTGCCCGGCCGGGTGCAGCCTGACCGCGGGTACTGTCTGGGG
TACCGCCATCTATCATGAACTTTCGTCCGTCTGCCGGGCCGCCATTCACGCCGGAAAGGTCCCG
AATAGCGGCGGTGCAGTGCACGTCGTGAACAACGGACCCTACTCGGATTTCCTCGCCTCGGACT
TGAATGGCATTAAGTCCGACGAGCTGAAGTCCCTGGCCCAGTCCTTCCGGTTCGACTACGTGTC
ATCCAGCACGGCCGGACGGAAGTCGGGCTGCCCTGATGGCTGGTTCGAAATCGAAGAGAATTGT
GTCTACGTGACCTCAAAGCAGAGAGCCTGGGAACGGGCTCAGGGAGTCTGCACTAACATGGCTG
CCCGGTTGGCCGTGCTCGACAAGGATGTGATCCCGTCCTCGCTCACTGAAACCCTGCGGGGAAA
GGGACTGGCTACCACTTGGATCGGACTGCACAGGCTCGATGCGGACAACCACTTCATCTGGGAA
CTGATGGACCGCTCCTCCGTGGCCCTGAACGACAGCCTGACCTTCTGGGCCCCTGGAGAGCCAG
GAAACGAAACCAACTGCGTGTACCTGGACATCCAGGACCAACTGCAGCCTGTCTGGAAAACCAA
GTCGTGCTTTCAACCCTCCTCTTTCGTGTGCATGATGGACCTGAGCGATAAGAACAAGGCCAAG
TGCAAGGATCCCGGCCCGCTCGAGAACGGTCACGCGAAGCTGCACGGGCAGTCCATCGACGGCT
TCTATGCCGGTTCCTCCGTGCGCTACTCGTGTGAAGTGTTGCACTACCTGTCCGGTACCGAAAC
CGTGTCCTGTACCTCCAACGGGACTTGGTCGGCCCCGAAGCCACGCTGTATTAAGGTCATCACC
TGTCAGACTCCGCCTGTCCCGTCCTACGGGTCCGTGGATATCAAGCCCCCGAGCCGGACTAACT
CGATTAGCCGCGTGGGCTCACCCTTTCTGCGGCTCCCAAGGTTGCCGCTTCCGCTGGCCCGGGC
GGCCGGCCCGCCTCCGAAGCCTAGATCCGCGCCTCCGTCCACCGTGGATTTGTCCAGCAAAGTC
AAGCTCCCTGAGGGACATTACAGAGTGGGATCCCAGGCCATCTACACTTGCGAATCACGCTATT
ACGAGCTGCTCGGATCGCAAGGCAGACGCTGTGACAGCAACGGAAAGTGGTCGGGCAGACCGGC
CAGCTGTATTCCCGTGTGCGGGGGGAGCGACTCGCCTCGCTCCCCCTTCATCGTGAACGGAAAC
TCAACCGAAATCGGCCAGTGGCCGTGGCAGGCCGGAATCTCTCGGTGGCTGGCTGATCATAACA
TGTGGTTCCTGCAATGCGGGGGCGCCCTGCTGAACGAGAAGTGGATAATCACAGCCGCTCACTG
CGTGACATACTCGGCAACCGCCGAAATCATCGATCCCTCCCAATTCAAGTTCTACCTGGGAAAG
TACTACCGGGATGACTCTAAGGACGACGATTATGTGCAAGTCCGGGAAGCCATTGAGATCCACG
TGAACCCTAACTACGATCCGGGAAATCTGAACTTCGACATTGCGCTGATCCAGCTGAAAACCTC
CGTGGCGCTGACCACTCGCGTCCAGCCGATCTGCCTCCCCACTGACCTGACCACACGGGAGAAC
CTGAAGGAAGGGGCCCTGGCAGTCGTGACCGGATGGGGACTCAACGAGAACAACACCTACTCCG
AGATGATCCAGCAGGCCGTGCTCCCTGTGGTGGCGGCCAGCACCTGTGAACAGGGCTACCAGGA
CTCCGGCCTCCCACTGACTGTGACTGAGAACATGTTCTGTGCCGGGTACAAGCAGGGGCGCTAC
GACGCGTGTTCCGGCGACAGCGGGGGGCCACTGGTGTTCGCCGACGACTCGAGGACCGACCGGC
GCTGGGTGCTCGAGGGTATTGTGTCCTGGGGATCCCCCAACGGATGCGGGAAGTCCAACCAATA
CGGCGGATTCACCAAGGTCAACGTGTTCTTGTCCTGGATTCGCCAGTTCATCTGA
SEQ ID NO: 2
RGVDLGLCDDTRFECKCGDPGYVFNVPAKQCTYFYRWRPYCKPCDKLEAKDVCPKYKRCQECRA
GLDSCVSCPPNKYGTWCSGECQCKNGGICDQRTGACTCRDRYEGVHCEILQGCPLLQSDPQVQE
VKNPPNDPQTIDYSCSPGFKLKGVARITCLPNGQWSSFPPKCIRECSMVSSLEHGKVNSPSADL
IRGATLRFSCDSPYYLIGQETLTCQGNGQWSGQIPQCQKLVFCPDLDPVSHARHQVKIGLEQKY
GQFPQGTEVTYTCTGNYFLMGLDTLKCNPDGSWSGTQPSCVKVADREVNCDSKAVDFLDDVGEP
VRIHCPAGCSLTAGTVWGTAIYHELSSVCRAAIHAGKVPNSGGAVHVVNNGPYSDFLASDLNGI
KSDELKSLAQSFRFDYVSSSTAGRKSGCPDGWFEIEENCVYVTSKQRAWERAQGVCTNMAARLA
VLDKDVIPSSLTETLRGKGLATTWIGLHRLDADNHFIWELMDRSSVALNDSLTFWAPGEPGNET
NCVYLDIQDQLQPVWKTKSCFQPSSFVCMMDLSDKNKAKCKDPGPLENGHAKLHGQSIDGFYAG
SSVRYSCEVLHYLSGTETVSCTSNGTWSAPKPRCIKVITCQTPPVPSYGSVDIKPPSRTNSISR
VGSPFLRLPRLPLPLARAAGPPPKPRSAPPSTVDLSSKVKLPEGHYRVGSQAIYTCESRYYELL
GSQGRRCDSNGKWSGRPASCIPVCGRSDSPRSPFIVNGNSTEIGQWPWQAGISRWLADHNMWFL
QCGGALLNEKWIITAAHCVTYSATAEIIDPSQFKFYLGKYYRDDSKDDDYVQVREAIEIHVNPN
YDPGNLNFDIALIQLKTSVALTTRVQPICLPTDLTTRENLKEGALAVVTGWGLNENNTYSEMIQ
QAVLPVVAASTCEQGYQDSGLPLTVTENMFCAGYKQGRYDACSGDSGGPLVFADDSRTDRRWVL
EGIVSWGSPNGCGKSNQYGGFTKVNVFLSWIRQFI
SEQ ID NO: 3 (Residues 1-25 shown in bold represent the signal sequence)
MVLASFLVSGLVLGLLAQKMRPVQSRGVDLGLCDDTRFECKCGDPGYVFNVPAKQCTYFYRWRP
YCKPCDKLEAKDVCPKYKRCQECRAGLDSCVSCPPNKYGTWCSGECQCKNGGICDQRTGACTCR
DRYEGVHCEILQGCPLLQSDPQVQEVKNPPNDPQTIDYSCSPGFKLKGVARITCLPNGQWSSFP
PKCIRECSMVSSLEHGKVNSPSADLIEGATLRFSCDSPYYLIGQETLTCQGNGQWSGQIPQCQK
LVFCPDLDPVSHAEHQVKIGLEQKYGQFPQGTEVTYTCTGNYFLMGLDTLKCNPDGSWSGTQPS
CVKVADREVNCDSKAVDFLDDVGEPVRIHCPAGCSLTAGTVWGTAIYHELSSVCRAAIHAGKVP
NSGGAVHVVNNGPYSDFLASDINGIKSDELKSLAQSFRFDYVSSSTAGRKSGCPDGWFEIEENC
VYVTSKQRAWERAQGVCTNMAARLAVLDKDVIPSSLTETLRGKGLATTWIGLHRLDADNHFIWE
LMDRSSVALNDSLTFWAPGEPGNETNCVYLDIQDQLQPVWKTKSCFQPSSFVCMMDLSDKNKAK
CKDPGPLENGHAKLHGQSIDGFYAGSSVRYSCEVLHYLSGTETVSCTSNGTWSAPKPRCIKVIT
CQTPPVPSYGSVDIKPPSRTNSISRVGSPFLRLPRLPLPLARAAGPPPKPRSAPPSTVDLSSKV
KLPEGHYRVGSQAIYTCESRYYELLGSQGRRCDSNGKWSGRPASCIPVCGRSDSPRSPFIVNGN
STEIGQWPWQAGISRWLADHNMWFLQCGGALLNEKWIITAAHCVTYSATAEIIDPSQFKFYLGK
YYRDDSKDDDYVQVREAIEIHVNPNYDPGNLNFDIALIQLKTSVALTTRVQPICLPTDLTTREN
LKEGALAVVTGWGLNENNTYSEMIQQAVLPVVAASTCEQGYQDSGLPLTVTENMFCAGYKQGRY
DACSGDSGGPLVFADDSRTDRRWVLEGIVSWGSPNGCGKSNQYGGFTKVNVFLSWIRQFI
SEQ ID NO: 4
ATGGCCTGGATCTGTGTCATTACCCTTTTCGCTCTCGCCTCGTCTACTCTGTCCAACAAAGTGT
CGAGGGTCGGAATCATCTTCCCCAAGACTCAGAACGACAACAAGCAGTGCACGGCTAAGGGTGG
ACTCAAGGGCAGCTGCAAATCCCTCACTGACTGCCCCGCCGTGCTGGCGACCCTTAAGGACAGC
TTCCCTGTCGTGTGTAGCTGGAATGGCCGGTTCCAACCGATTGTGTGCTGTCCGGACGCCGCCG
CCCCTTCCGTGACAACTACCGTGACCACTATCGTGCCGACCAAGGAAACTAAGATCCCTAGACT
GCATATCCCGGGCTGCGGGAAACGGAAAGTCAACGTGGACATCACCACCATCGGACGGTCCGGT
AGCCCGATCCTGCCGCCCATTAGCACGTCACAGGACCTGAAGGGGGGCCGCGGAATCATTGCCG
GCGGAGTGGAAGCGAAGATTGGGGCGTGGCCCTGGATGGCCGCAGTGTTCGTGAAGAACTTTGG
CATTGGAAGATTCCACTGCGCGGGATCCATCATTTCGTCCAAGTACATTCTGTCCGCCGCCCAC
GCCTTCCTGATCGGCGGCCGCAAGCTGACCCCCACCCGCTTGGCCGTGCGCGTGGGCGGACACT
ACGTCAAGATGGGCCAAGAGTACCACGTGGAAGATGTCATCATCCACCCTGACTACGTGGAACG
GGAGAACTACAACGACATCGCTATCATCGTGCTGAAGGAGGAGCTGAACTTCACCGACCTCGTG
CGCCCAATCTGCCTGCCGGATCCAGAGGCCGTGACCGATTCACTCAAGGGTCGGAGAGTGACCG
TCGCCGGTTGGGGGGACCTCGACTTCGCGGGCCCCCGGTCCCAAGTGCTGCGGGAGGTGTCCAT
ACCAGTGGTGCCTATTGGCGATTGCAACAAGGCATATCAGAAGCTCAACACCTTGGCTCTGAAG
AACGGAATTACCAAGAAGTTCATCTGCGCCGGGCTGGAAGAAGGGGGAAAGGACGCGTGCCAGG
GCGATTCGGGTGGACCCCTGATGCTGGTCAACAACTCATCGTGGATCGTGACCGGAGTGGTGTC
CTTCGGACATAAGTGCGCCGAAGAAGGGTTTCCGGGAGTCTACACCAGGGTGGTGTCCTACCTG
GAGTGGATCGCAAAGGTCACCAATTCCTTGGATCAGACCGTCACTAACTAA
SEQ ID NO: 5
IIFPKTQNDNKQCTAKGGLKGSCKSLTDCPAVLATLKDSFPVVCSWNGRFQPIVCCPDAAAPSV
TTTVTTIVPTKETKIPRLHIPGCGKRKVNVDITTIGRSGSPILPPISTSQDLKGGRGIIAGGVE
AKIGAWPWMAAVFVKNFGIGRFHCAGSIISSKYILSAAHAFLIGGRKLTPTRLAVRVGGHYVKM
GQEYHVEDVIIHPDYVERENYNDIAIIVLKEELNFTDLVRPICLPDPEAVTDSLKGRRVTVAGW
GDLDFAGPRSQVLREVSIPVVPIGDCNKAYQKLNTLALKNGITKKFICAGLEEGGKDACQGDSG
GPLMLVNNSSWIVTGVVSFGHKCAEEGFPGVYTRVVSYLEWIAKVINSLDQTVTN
SEQ ID NO: 6 (Residues 1-25 shown in bold represent the signal sequence)
MAWICVITLFALASSTLSNKVSRVGIIFPKTQNDNKQCTAKGGLKGSCKSUTDCPAVLATLKDS
FPVVCSWNGRFQPIVCCPDAAAPSVTTTVTTIVPTKETKIPRLHIPGCGKRKVNVDITTIGRSG
SPILPPISTSQDLKGGRGIIAGGVEAKIGAWPWMAAVFVKNFGIGRFHCAGSIISSKYILSAAH
AFLIGGRKLTPTRLAVRVGGHYVKMGQEYHVEDVIIHPDYVERENYNDIAIIVLKEELNFTDLV
RPICLPDPEAVTDSLKGRRVTVAGWGDLDFAGPRSQVLREVSIPVVPIGDQNKAYQKLNTLALK
NGITKKFICAGLEEGGKDACQGDSGGPLMLVNNSSWIVTGVVSFGHKCAEEGFPGVYTRVVSYL
EWIAKVINSLDQTVTN
SEQ ID NO: 7
ATGGGAATCTTGCCCTCGCCCGGAATGCCTGCCCTGCTTAGCCTCGTGTCACTCCTGTCCGTCC
TGCTCATGGGCTGCGTGGCCTCCTCACTGGGACGCCAGCGGAGACAGTTCGTGTTCCCGGATGA
CGAAGAGTCCTGTAGCAACCGGTTTACCAACGACGGGATCTGCAAGGACGTGCTGAACTGCCGC
GATCTGCTGCAGAAGAATGACTACAACCTCTTGAAGGAATCCATCTGCGGCTTCGAAGGGATCA
CCCCTAAAGTCTGCTGTCCAAAGCAATCCATTGTGAACCCCATCACCGAAGCCCCGCCTAAGAC
CACCACTACTGAGAGGCCTCCGATCCGGATCCCCTCCAACCTCCCGAAGCAGTGCGGAAATCGG
AACATCACTACCACTAGGATCATCGGCGGACAGGAAGCCACTCCGGGAGCCTGGCCGTGGATGG
CAGCCGTGTACATTAAGCAGGGGGGGATTCGGTCCGTGCAGTGCGGTGGCGCCCTTGTGACCAA
CAGACACGTGATCACCGCCTCACACTGCGTCGTGAATTCCCTGGGTACCGATGTGATGAGGGCT
GATGTGTTCTCCGTGCGGCTGGGAGAACATAACCTGTACAGCACTAACGACTCGTCAGACCCAA
TCGACTTCGCCGTGACGAGCGTGAAGCACCATGAAAACTTCGTGCTGGCCACCTACCTCAACGA
CATCGCGATCCTGAAGCTGAACGACACTGTCACCTTCACACACAAGATTAAGCCGATTTGCCTC
CCTTACGAGTCCCTGCGCTATGAGGACCTCGCTATGCGCAACCCCTTCGTGGCCGGATGGGGCA
CTACCGCGTTCAACGGACCGAGCAGCGCTGTCCTGAGAGAAGTGCAACTCCCAATCTGGGGTCA
CGAGCCGTGCCGCCAAGCGTACGAGAAAGACCTGAACATCACCAACGTGTATATGTGGGGGGGG
TACGCCGACGGTGGAAAGGACGCATGCCAGGGCGATTCTGGGGGCCCCATGATGCTGCCTGACA
AGTCCGGGAACTTCTACCTGGTCGGAATCGTGTCGTTCGGAAAGAAATGCGCCCTGCCCGGCTT
TCCCGGAGTGTACACCAAGGTCACCGAATTTTTGGATTGGATTGCCGTGAACATGGTCTAA
SEQ ID NO: 8
SSLGRQRRQFVFPDDEESCSNRFTNDGICKDVLNCRDLLQKNDYNLLKESICGFEGITPKVCCP
KQSIVNPITEAPPKTTTTERPPIRIPSNLPKQCGNRNITTTRIIGGQEATPGAWPWMAAVYIKQ
GGIRSVQCGGALVTNRHVITASHCVVNSLGTDVMRADVFSVRLGEHNLYSTNDSSDPIDFAVTS
VKHHENFVLATYLNDIAILKENDTVTFTHKIKPICLPYESLRYEDLAMRNPFVAGWGTTAFNGP
SSAVLREVQLPIWGHEPCRQAYEKDLNITNVYMCAGYADGGKDACQGDSGGPMMLPDKSGNFYL
VGIVSFGKKCALPGFPGVYTKVTEFLDWIAVNMV
SEQ ID NO: 9 (Residues 1-28 shown in bold represent the signal sequence)
MGILPSPGMPALLSLVSLLSVLLMGCVASSLGRQRRQFVFPDDEESCSNRFTNDGICKDVLNCR
DLLQKNDYNLLKESICGFEGITPKVCCPKQSIVNPITEAPPKTTTTERPPIRIPSNLPKQCGNR
NITTTRIIGGQEATPGAWPWMAAVYIKQGGIRSVQCGGALVTNRHVITASHCVVNSLGTDVMRA
DVFSVRLGEHNLYSTNDSSDPIDFAVTSVKHHENFVLATYLNDIAILKLNDTVTFTHKIKPICL
PYESLRYEDLAMRNPFVAGWGTTAFNGPSSAVLREVQLPIWGHEPCRQAYEKDLNITNVYMCAG
YADGGKDACQGDSGGPMMLPDKSGNFYLVGIVSFGKKCALPGFPGVYTKVTEFLDWIAVNMV
SEQ ID NO: 10
ATGAAGTTCCTCGTCAACGTCGCCCTCGTGTTTATGGTGGTGTATATTAGCTACATCTATGCTC
GTGGCGTCGATCTGGGCCTGTGCGATGAGACCCGCTTCGAATGTAAGTGCGGCGACCCCGGCTA
TGTGTTCAACGTCCCCATGAAGCAGTGCACCTACTTCTACCGCTGGCGCCCCTACTGTAAGCCT
TGCGATGACCTGGAAGCTAAAGATATTTGCCCTAAGTATAAGCGCTGCCAAGAATGTAAGGCTG
GTCTGGATTCCTGCGTCACCTGTCCCCCTAACAAGTATGGCACCTGGTGTTCCGGTGAATGCCA
GTGCAAAAACGGCGGCATCTGCGATCAGCGTACTGGCGCTTGCACCTGCCGTGATCGCTACGAG
GGCGCCCACTGTGAAATTCTGAAAGGCTGTCCTCTGCTGCCTTCCGATAGCCAAGTGCAGGAGG
TGCGTAACCCCCCTGACAACCCTCAGACCATCGATTATAGCTGCTCCCCCGGCTTCAAGCTGAA
GGGCGTGGCCCGTATTAGCTGTCTGCCCAACGGTCAGTGGAGCTCCTTTCCCCCCAAGTGCATC
CGTGAATGTGCCAAGGTCTCCTCCCCCGAACACGGCAAGGTCAACGCTCCCTCCGGCAACATGA
TCGAAGGCGCCACCCTCCGTTTTTCCTGCGACTCCCCTTACTACCTCATCGGCCAGGAGACCCT
GACTTGTCAGGGCAATGGCCAGTGGAGCGGTCAAATCCCTCAGTGCAAGAAGCTGGTGTTCTGT
CCTGACCTCGATCCCGTGAACCACGCTGAGCACCAAGTGAAGATCGGCGTGGAACAAAAGTACG
GTCAGTTCCCTCAGGGCACTGAGGTCACTTACACTTGCTCCGGCAACTACTTCCTGATGGGCTT
CAACACCCTGAAATGTAACCCCGACGGTTCCTGGTCCGGTTCCCAGCCCTCCTGCGTGAAAGTC
GCTGACCGTGAAGtcgaCTGTGACAGCAAAGCCGTGGACTTCCTGGACGACGTGGGCGAGCCTG
TGCGTATTCACTGTCCCGCTGGCTGTAGCCTGACTGCCGGTACCGTGTGGGGTACTGCTATCTA
CCACGAGCTCTCCAGCGTGTGCCGCGCTGCTATCCACGCTGGCAAGCTGCCTAACAGCGGCGGT
GCTGTCCACGTGGTGAACAACGGTCCCTACTCCGACTTCCTCGGTAGCGACCTGAACGGTATCA
AAAGCGAGGAACTGAAGTCCCTGGCCCGTAGCTTTCGCTTCGACTACGTGTCCTCCTCCACCGC
TGGCCGTAGCGGCTGTCCTGATGGCTGGTTTGAGGTGGAAGAGAATTGTGTGTACGTGACCTCC
AAGCAGCGTGCTTGGGAGCGTGCTCAGGGTGTCTGTACCAATATGGCCGCCCGTCTGGCTGTCC
TCGATAAGGATCTCATCCCTTCCTCCCTCACCGAGACTCTGCGCGGTAAGGGCCTGACCACTAC
CTGGATCGGTCTCCACCGCCTGGACGCTGAGAAGCCTTTCGTCTGGGAGCTCATGGACCGCAGC
AACGTGGTGCTGAACGACAACCTGACCTTCTGGGCCAGCGGCGAGCCTGGCAATGAAACCAACT
GCGTGTACCTGGACATCCGTGATCAGCTCCAGCCCGTGTGGAAGACCAAGAGCTGCTTCCAGCC
CTCCAGCTTTGCTTGCATGATGGATCTCTCCGACCGCAACAAGGCTAAATGTGATGATCCCGGC
CCCCTGGAGAACGGCCATGCCACTCTGCATGGCCAGAGCATCGACGGTTTCTACGCTGGTTCCT
CCATCCGCTACTCCTGCGAAGTGCTCCACTACCTGTCCGGCACCGAGACCGTGACCTGTACTAC
CAACGGTACTTGGAGCGCTCCTAAACCCCGCTGTATCAAGGTGATTACCTGTCAGAACCCTCCT
GTCCCCAGCTACGGCTCCGTGGAAATCAAGCCCCCCAGCCGTACTAATAGCATCAGCCGTGTCG
GTTCCCCTTTCCTGCGTCTGCCTCGTCTGCCCCTGCCTCTCGCTCGCGCTGCTAAGCCTCCCCC
TAAGCCCCGTTCCAGCCAGCCTAGCACTGTGGATCTCGCTAGCAAAGTGAAACTCCCCGAGGGT
CACTATCGTGTGGGCTCCCGCGCCATTTACACCTGCGAGAGCCGCTACTACGAGCTCCTGGGCA
GCCAGGGTCGTCGTTGCGACAGCAACGGTAACTGGAGCGGTCGCCCTGCCAGCTGCATTCCCGT
GTGCGGTCGCAGCGACTCCCCTCGCAGCCCTTTCATCTGGAACGGCAACAGCACCGAGATCGGT
CAGTGGCCCTGGCAAGCTGGCATTAGCCGCTGGCTCGCTGATCACAACATGTGGTTCCTCCAAT
GTGGCGGCAGCCTGCTGAACGAGAAGTGGATCGTGACTGCCGCTCACTGCGTGACCTACAGCGC
CACCGCTGAGATCATCGACCCCTCCCAATTTAAGATCTACCTCGGCAAGTACTACCGCGACGAT
AGCCGCGACGACGATTATGTGCAAGTCCGCGAAGCTCTCGAAATTCACGTGAACCCTAACTACG
ATCCCGGTAACCTGAACTTCGACATTGCCCTGATCCAGCTGAAGACCCCTGTGACCCTCACCAC
TCGCGTCCAGCCTATCTGCCTCCCCACCGACATTACCACTCGCGAACATCTGAAAGAAGGTACC
CTGGCCGTCGTGACCGGCTGGGGCCTCAACGAAAACAACACTTACAGCGAGATGATCCAGCAGG
CTGTGCTGCCTGTCGTGGCTGCTAGCACCTGTGAGGAAGGCTACAAAGAGGCCGATCTCCCCCT
GACCGTCACCGAGAACATGTTTTGCGCTGGTTACAAAAAGGGCCGCTACGACGCCTGCAGCGGT
GATAGCGGTGGCCCTCTCGTGTTCGCCGATGATTCCCGTACCGAACGTCGCTGGGTGCTCGAGG
GCATTGTGTCCTGGGGTAGCCCTTCCGGCTGTGGCAAGGCTAACCAATACGGGGGCTTCACCAA
GGTGAACGTGTTCCTGTCCTGGATTCGTCAGTTCATCTAATGA
SEQ ID NO: 11
RGVDLGLCDETRFECKCGDPGYVFNVPMKQCTYFYRWRPYCKPCDDLEAKDICPKYKRCQECKA
GLDSCVTCPPNKYGTWCSGECQCKNGGICDQRTGACTCRDRYEGAHCEILKGCPLLPSDSQVQE
VRNPPDNPQTIDYSCSPGFKLKGVARISCLPNGQWSSFPPKCIRECAKVSSPEHGKVNAPSGNM
IEGATLRFSCDSPYYLIGQETLTCQGNGQWSGQIPQCKKLVFCPDLDPVNHAEHQVKIGVEQKY
GQFPQGTEVTYTCSGNYFLMGFNTLKCNPDGSWSGSQPSCVKVADREVDCDSKAVDFLDDVGEP
VRIHCPAGCSLTAGTVWGTAIVHELSSVCRAAIHAGKLPNSGGAVHVVNNGPYSDFLGSDLNGI
KSEELKSLARSFRFDYVSSSTAGRSGCPDGWFEVEENCVYVTSKQRAWERAQGVCTNMAARLAV
LDKDLIPSSLTETLRGKGLTTTWIGLHRLDAEKPFVWELMDRSNVVLNDNLTFWASGEPGNETN
CVYLDIRDQLQPVWKTKSCFQPSSFACMMDLSDRNKAKCDDPGPLENGHATLHGQSIDGFYAGS
SIRYSCEVLHYLSGTETVTCTTNGTWSAPKPRCIKVITCQNPPVPSYGSVEIKPPSRTNSISRV
GSPFLRLPRLPLPLARAAKPPPKPRSSQPSTVDLASKVKLPEGHYRVGSRAIYTCESRYYELLG
SQGRRCDSNGNWSGRPASCIPVCGRSDSPRSPFIWNGNSTEIGQWPWQAGISRWLADHNMWFLQ
CGGSLLNEKWIVTAAHCVTYSATAEIIDPSQFKIYLGKYYRDDSRDDDYVQVREALEIHVNPNY
DPGNLNFDIALIQLKTPVTLTTRVQPICLPTDITTREHLKEGTLAVVTGWGLNENNTYSEMIQQ
AVLPVVAASTCEEGYKEADLPLTVTENMFCAGYKKGRYDACSGDSGGPLVFADDSRTERRWVLE
GIVSWGSPSGCGKANQYGGFTKVNVFLSWIRQFI
SEQ ID NO: 12 (Residues 1-21 in bold represent the signal sequence)
MKFLVNVALVFMVVYISYIYARGVDLGLCDETRFECKCGDPGYVFNVPMKQCTYFYRWRPYCKP
CDDLEAKDICPKYKRCQECKAGLDSCVTCPPNKYGTWCSGECQCKNGGICDQRTGACTCRDRYE
GAHCEILKGCPLLPSDSQVQEVRNPPDNPQTIDYSCSPGFKLKGVARISCLPNGQWSSFPPKCI
RECAKVSSPEHGKVNAPSGNMIEGATLRFSCDSPYYLIGQETLTCQGNGQWSGQIPQCKKLVFC
PDLDPVNHAEHQVKIGVEQKYGQFPQGTEVTYTCSGNYFLMGFNTLKCNPDGSWSGSQPSCVKV
ADREVDCDSKAVDFLDDVGEPVRIHCPAGCSLTAGTVWGTAIYHELSSVCRAAIHAGKLPNSGG
AVHVVNNGPYSDFLGSDLNGIKSEELKSLARSFRFDYVSSSTAGRSGCPDGWFEVEENCVYVTS
KQRAWERAQGVCTNMAARLAVLDKDLIPSSLTETLRGKGLTTTWIGLHRLDAEKPFVWELMDRS
NVVLNDNLTFWASGEPGNETNCVYLDIRDQLQPVWKTKSCFQPSSFACMMDLSDRNKAKCDDPG
PLENGHATLHGQSIDGFYAGSSIRYSCEVLHYLSGTETVTCTTNGTWSAPKPRCIKVITCQNPP
VPSYGSVEIKPPSRTNSISRVGSPFLRLPRLPLPLARAAKPPPKPRSSQPSTVDLASKVKLPEG
HYRVGSRAIYTCESRYYELLGSQGRRCDSNGNWSGRPASCIPVCGRSDSPRSPFIWNGNSTEIG
QWPWQAGISRWLADHNMWFLQCGGSLLNEKWIVTAAHCVTYSATAEIIDPSQFKIYLGKYYRDD
SRDDDYVQVREALEIHVNPNYDPGNLNFDIALIQLKTPVTLTTRVQPICLPTDITTREHLKEGT
LAVVTGWGLNENNTYSEMIQQAVLPVVAASTCEEGYKEADLPLTVTENMFCAGYKKGRYDACSG
DSGGPLVFADDSRTERRWVLEGIVSWGSPSGCGKANQYGGFTKVNVFLSWIRQFI
SEQ ID NO: 13
ATGGACATGCGCGTGCCTGCCCAACTGCTCGGCCTGCTGCTGCTGTGGTTCCCGGGTAGCCGCT
GTGTGGGGGTGCTGTTCCCCAAGACCCGGAACGACAACGAATGCACTGCCAGGGGTGGATTGAA
GGGGTCCTGCAAGAGCCTGATCGACTGCCCGTCGGTGCTTGCCACCCTGAAGGACTCCTTTCCT
GTCGTGTGCTCCTGGAACGGCAGATTCCAGCCAATTGTGTGCTGTCCGGATGCAATTGCCCCCC
CACCTGTGACCACCACCGCCGTGACCGTGATCTCTACCAAGGAACCCAAGCTGCCTCGGCTCCA
CATCAGCGGTTGTGGGAAGCGGAAGGTCAAGATTGACATTACCACTGTGGGACGCTCAGGATCA
CCGATTCTGCCCCCGATCTCCACCCCGCAAAACTCCACTGGTGGCAGGGGCATCATTGCCGGTG
GAGTGGAAGCGAAGATCGGAGCCTGGCCTTGGATGGCGGCGGTGTTTGTGAAAAACTTCGGAAT
CGGGCGGTTCCACTGCGCTGGTTCGATCATCTCCAACAAGTACATCCTGTCCGCCGCACATGCC
TTCCTGATCGGAGGCCGGAAGTTGACCCCCACGAGACTGGCCGTCAGAGTCGGAGGGCATTACA
TCAAGCGCGGACAGGAATACCCCGTGAAGGACGTGATAATCCACCCCCACTACGTGGAAAAGGA
GAACTACAATGACATCGCCATTATCGAGCTGAAGGAGGAGCTGAACTTTACCGATCTTGTGAAC
CCCATCTGCCTCCCTGACCCCGAGACAGTGACCGACCCACTCAAAGACCGCATCGTGACTGCAG
CTGGATGGGGCGATCTGGACTTCAGCGGACCGAGAAGCCAGGTCCTGCGGGAGGTGTCAATCCC
GGTGGTGCCAGTCGACAAATGCGATCAGGCCTACGAGAAGCTGAACACTCCCTCCCTCAAAAAC
GGGATCACTAACAACTTCCTGTGCGCGGGACTTGAAGAAGGCGGAAAGGACGCCTGTCAGGGCG
ATTCCGGCGGACCTCTGATGCTCGTGAACAATACTCGGTGGATTGTCGTCGGCGTGGTGTCCTT
CGGACATAAGTGCGCCGAGGAAGGCTACCCTGGCGTCTATTCCCGCGTGGCCTCCTACCTGGAC
TGGATTGCCAAGGTCACCAACTCGCTCGACCATGCCGTGACTAACTGA
SEQ ID NO: 14
VGVLFPKTRNDNECTARGGLKGSCKSLIDCPSVLATLKDSFPVVCSWNGRFQPIVCCPDAIAPP
PVTTTAVTVISTKEPKLPRLHISGCGKRKVKIDITTVGRSGSPILPPISTPQNSTGGRGIIAGG
VEAKIGAWPWMAAVFVKNFGIGRFHCAGSIISNKYILSAAHAFLIGGRKLTPTRLAVRVGGHYI
KRGQEYPVKDVIIHPHYVEKENYNDIAIIELKEELNFTDLVNPICLPDPETVTDPLKDRIVTAA
GWGDLDFSGPRSQVLREVSIPVVPVDKCDQAYEKLNTPSLKNGITNNFLCAGLEEGGKDACQGD
SGGPLMLVNNTRWIVVGVVSFGHKCAEEGYPGVYSRVASYLDWIAKVTNSLDHAVTN
SEQ ID NO: 15 (Residues 1-22 represent the signal sequence)
MDMRVPAQLLGLLLLWFPGSRCVGVLFPKTRNDNECTARGGLKGSCKSLIDCPSVLATLKDSFP
VVCSWNGRFQPIVCCPDAIAPPPVTTTAVTVISTKEPKLPRLHISGCGKRKVKIDITTVGRSGS
PILPPISTPQNSTGGRGIIAGGVEAKIGAWPWMAAVFVKNFGIGRFHCAGSIISNKYILSAAHA
FLIGGRKLTPTRLAVRVGGHYIKRGQEYPVKDVIIHPHYVEKENYNDIAIIELKEELNFTDLVN
PICLPDPETVTDPLKDRIVTAAGWGDLDFSGPRSQVLREVSIPVVPVDKCDQAYEKLNTPSLKN
GIINNFLCAGLEEGGKDACQGDSGGPLMLVNNTRWIVVGVVSFGHKCAEEGYPGVYSRVASYLD
WIAKVTNSLDHAVTN
SEQ ID NO: 16
ATGCTGGTCAACAATGTCTTTTCCCTGCTCTGCTTCCCTCTCCTGATGTCCGTCGTGCGCTGCT
CCACCCTCTCGAGACAGCGGCGCCAGTTTGTGTTCCCTGATGAAGAGGAACTGTGTTCCAACCG
GTTCACCGAAGAGGGAACTTGCAAGAACGTGCTGGACTGCCGCATCCTGCTTCAAAAGAACGAC
TACAACCTTCTCAAGGAGTCAATCTGCGGTTTCGAAGGGATTACCCCAAAAGTCTGCTGCCCGA
AGTCCTCGCACGTGATCTCAAGCACCCAGGCACCCCCTGAAACTACCACTACTGAGCGGCCCCC
GAAGCAAATTCCCCCGAACTTGCCGGAAGTCTGCGGCATCCACAACACCACCACCACTAGGATC
ATCGGGGGGAGAGAAGCCCCTATCGGCGCCTGGCCCTGGATGACCGCTGTGTACATTAAGCAGG
GCGGTATCCGGAGCGTGCAGTGCGGTGGAGCTCTGGTCACCAATCGCCACGTGATCACAGCGTC
ACACTGCGTCGTGAACTCGGCGGGTACCGACGTGATGCCCGCCGACGTGTTCTCTGTGCGGTTG
GGGGAACATAACCTTTACTCGACCGATGATGACTCCAACCCAATCGATTTCGCCGTGACGTCCG
TGAAGCACCATGAGCACTTCGTGCTGGCCACCTACCTGAACGACATAGCAATTCTCACTCTGAA
CGACACCGTGACTTTTACTGATCGGATCAGGCCAATCTGTCTGCCGTACCGCAAGCTCAGATAC
GACGACCTGGCCATGCGCAAGCCGTTCATTACGGGTTGGGGAACTACCGCCTTCAACGGACCTT
CCTCCGCCGTGCTGCGGGAAGTGCAGCTGCCGATCTGGGAGCACGAAGCCTGTAGACAAGCCTA
CGAAAAGGACCTGAACATTACCAACGTGTATATGTGTGCCGGATTCGCGGATGGCGGCAAAGAC
GCATGCCAGGGAGACTCCGGCGGCCCGATGATGCTGCCTGTGAAAACCGGAGAGTTCTACCTCA
TCGGCATCGTCAGCTTCGGGAAGAAATGCGCCCTGCCCGGATTCCCCGGAGTGTACACTAAGGT
CACCGAGTTCCTCGACTGGATTGCCGAACACATGGTGTAA
SEQ ID NO: 17
STLSRQRRQFVFPDEEELCSNRFTEEGTCKNVLDCRILLQKNDYNLLKESICGFEGITPKVCCP
KSSHVISSTQAPPETTTTERPPKQIPPNLPEVCGIHNTTTTRIIGGREAPIGAWPWMTAVYIKQ
GGIRSVQCGGALVTNRHVITASHCVVNSAGTDVMPADVFSVRLGEHNLYSTDDDSNPIDFAVTS
VKHHEHFVLATYLNDIAILTLNDTVTFTDRIRPICLPYRKLRYDDLAMRKPFITGWGTTAFNGP
SSAVLREVQLPIWEHEACRQAYEKDLNITNVYMCAGFADGGKDACQGDSGGPMMLPVKTGEFYL
IGIVSFGKKCALPGFPGVYTKVTEFLDWIAEHMV
SEQ ID NO: 18 (Residues 1-21 represent the signal sequence)
MLVNNVFSLLCFPLLMSVVRCSTLSRQRRQFVFPDEEELCSNRFTEEGTCKNVLDCRILLQKND
YNLLKESICGFEGITPKVCCPKSSHVISSTQAPPETTTTERPPKQIPPNLPEVCGIHNTTTTRI
IGGREAPIGAWPWMTAVYIKQGGIRSVQCGGALVTNRHVITASHCVVNSAGTDVMPADVFSVRL
GEHNLYSTDDDSNPIDFAVTSVKHHEHFVLATYLNDIAILTLNDTVTFTDRIRPICLPYRKLRY
DDLAMRKPFITGWGTTAFNGPSSAVLREVQLPIWEHEACRQAYEKDLNITNVYMCAGFADGGKD
ACQGDSGGPMMLPVKTGEFYLIGIVSFGKKCALPGFPGVYTKVTEFLDWIAEHMV

What is claimed is:

1. A hybrid amebocyte lysate composition, the composition comprising:
(i) a native horseshoe crab amebocyte lysate; and
(ii) a recombinant horseshoe crab factor B and/or a recombinant horseshoe crab proclotting enzyme.

2. The composition of claim 1, wherein the composition comprises a recombinant horseshoe crab factor B and a recombinant horseshoe crab proclotting enzyme.

3. A hybrid amebocyte lysate composition, the composition comprising:
(i) a native horseshoe crab factor C;
(ii) a native horseshoe crab factor B;
(iii) a native horseshoe crab pro-clotting enzyme; and
(iv) a recombinant horseshoe crab factor B and/or a recombinant horseshoe crab proclotting enzyme.

4. The composition of claim 3, wherein the composition comprises a recombinant horseshoe crab factor B and a recombinant horseshoe crab proclotting enzyme.

5. A hybrid amebocyte lysate composition, the composition comprising:
(i) horseshoe crab factor C;
(ii) horseshoe crab factor B; and
(iii) horseshoe crab proclotting enzyme;
wherein (a) the ratio of horseshoe crab factor B to horseshoe crab factor C is greater than the ratio of horseshoe crab factor B to horseshoe crab factor C in a native horseshoe crab amebocyte lysate, and/or (b) the ratio of horseshoe crab proclotting enzyme to horseshoe crab factor C is greater than the ratio of horseshoe crab proclotting enzyme to horseshoe crab factor C in a native horseshoe crab amebocyte lysate.

6. The composition of claim 5, wherein the ratio of horseshoe crab factor B to horseshoe crab factor C is greater than the ratio of horseshoe crab factor B to horseshoe crab factor C in a native horseshoe crab amebocyte lysate, and the ratio of horseshoe crab proclotting enzyme to horseshoe crab factor C is greater than the ratio of horseshoe crab proclotting enzyme to horseshoe crab factor C in a native horseshoe crab amebocyte lysate.

7. The composition of any one of claims 1-6, wherein the composition does not comprise a recombinant horseshoe crab factor C.

8. The composition of any one of claims 1-7, wherein the composition comprises from about 0.05 to about 1 U/mL of recombinant horseshoe crab factor B.

9. The composition of claim 8, wherein the composition comprises from about 0.1 to about 0.5 U/mL of recombinant horseshoe crab factor B.

10. The composition of any one of claims 1-9, wherein the composition comprises from about 0.05 to about 2,000 U/mL of recombinant proclotting enzyme.

11. The composition of claim 10, wherein the composition comprises from about 50 to about 200 U/mL of recombinant proclotting enzyme.

12. The composition of any one of claims 1-11, wherein the composition has substantially the same sensitivity in detecting endotoxin as native horseshoe crab amebocyte lysate.

13. The composition of any one of claims 1-12, wherein the composition has a greater sensitivity in detecting endotoxin than native horseshoe crab amebocyte lysate.

14. The composition of any one of claims 1-13, wherein the horseshoe crab amebocyte lysate is a Limulus polyphemus or a Tachypleus amebocyte lysate.

15. The composition of any one of claims 1-14, wherein the horseshoe crab factor B is a Limulus polyphemus or a Tachypleus factor B.

16. The composition of any one of claims 1-15, wherein the horseshoe crab proclotting enzyme is a Limulus polyphemus or a Tachypleus proclotting enzyme.

17. The composition of any one of claims 1-16, wherein the horseshoe crab factor C is a Limulus polyphemus or a Tachypleus factor C.

18. The composition of any one of claims 1-17, wherein the recombinant horseshoe crab factor B and/or the recombinant horseshoe crab proclotting enzyme are expressed in a mammalian cell.

19. The composition of claim 18, wherein the mammalian cell is a Chinese hamster ovary (CHO) or human embryonic kidney (HEK) cell.

20. The composition of claim 19, wherein the recombinant factor B and/or the recombinant proclotting enzyme has different glycosylation than the factor B and/or the proclotting enzyme present in the native horseshoe crab amebocyte lysate.

21. A method for preparing an endotoxin detection reagent, the method comprising adding a recombinant horseshoe crab factor B and/or a recombinant horseshoe crab proclotting enzyme to a native horseshoe crab amebocyte lysate.

22. The method of claim 21, wherein the method comprises adding a recombinant horseshoe crab factor B and a recombinant horseshoe crab proclotting enzyme to the native horseshoe crab amebocyte lysate.

23. A method for increasing the endotoxin sensitivity of a native horseshoe crab amebocyte lysate, the method comprising adding to the native horseshoe crab amebocyte lysate a recombinant horseshoe crab factor B and/or a recombinant horseshoe crab proclotting enzyme, thereby to increase the endotoxin sensitivity of the native horseshoe crab amebocyte lysate.

24. The method of claim 23, wherein the method comprises adding a recombinant horseshoe crab factor B and a recombinant horseshoe crab proclotting enzyme to the native horseshoe crab amebocyte lysate.

25. A method for reducing the amount of a native horseshoe crab amebocyte lysate required to detect endotoxin, the method comprising: (i) diluting the native horseshoe crab amebocyte lysate; and (ii) adding to the diluted native horseshoe crab amebocyte lysate a recombinant horseshoe crab factor B and/or a recombinant horseshoe crab proclotting enzyme to produce a hybrid amebocyte lysate.

26. The method of claim 25, wherein the method comprises adding a recombinant horseshoe crab factor B and a recombinant horseshoe crab proclotting enzyme to the diluted native horseshoe crab amebocyte lysate.

27. The method of claim 25 or 26, wherein the hybrid amebocyte lysate has substantially the same, or greater, sensitivity in detecting endotoxin as the native horseshoe crab amebocyte lysate.

28. An isolated nucleic acid comprising the sequence of SEQ ID NO: 1, SEQ ID NO: 4, SEQ ID NO: 7, SEQ ID NO: 10, SEQ ID NO: 13, or SEQ ID NO: 16.

29. An expression vector comprising a nucleic acid of claim 28 operatively linked to a promoter.

30. A host cell (e.g., a mammalian host cell) comprising the expression vector of claim 29.

31. A method of producing a recombinant horseshoe crab factor C protein comprising:
(a) expressing a nucleic acid sequence encoding the recombinant horseshoe crab factor C in a host cell engineered to remove a glycosyltransferase enzyme; and
(b) purifying the recombinant factor C expressed in the host cell.

32. The method of claim 31, wherein the host cell is a HEK293 or CHO cell.

33. The method of claim 31 or 32, wherein the glycosyltransferase is N-acetylglucosaminyltransferase.

34. A recombinant horseshoe crab factor C protein produced by the method of any one of claims 31-33.

35. The recombinant factor C of claim 34, wherein the factor C does not contain (α-2,3)-linked terminal sialic acid.

36. A method of preparing an endotoxin detection reagent, the method comprising admixing the recombinant horseshoe crab factor C of claim 34 or 35 to a composition comprising recombinant horseshoe crab factor B and recombinant horseshoe crab proclotting enzyme.
